(19) United States
(12) Patent Application Publication
(10) Pub. No.: US 2022/0267770 A1
(43) Pub. Date: Aug. 25, 2022

(54) COMPOSITIONS AND METHODS OF USING C/EBP ALPHA SARNA

(71) Applicants: MiNA THERAPEUTICS LIMITED, London (GB); THE WISTAR INSTITUTE OF ANATOMY AND BIOLOGY, Philadelphia, PA (US)

(72) Inventors: Dmitry Gabrilovich, Villanova, PA (US); Robert Habib, London (GB); Vikash Reebye, London (GB)

(21) Appl. No.: 17/630,299

(22) PCT Filed: Jul. 27, 2020
(86) PCT No.: PCT/US20/43705

§ 371 Date: Jan. 26, 2022

Related U.S. Application Data
(60) Provisional application No. 62/879,028, filed on Jul. 26, 2019.
(60) Provisional application No. 63/050,091, filed on Jul. 9, 2020.

(51) Int. Cl. C12N 15/113; A61P 35/00; A61K 31/415; A61K 31/433; A61K 31/635; A61K 31/713

(52) U.S. Cl. CPC C12N 15/113; A61P 35/00; A61K 31/415; A61K 31/433; A61K 31/635; A61K 31/713; C12N 2310/14; C12N 2310/321; C12N 2310/332; C12N 2320/31

(57) ABSTRACT

The disclosure relates to a pharmaceutical composition comprising an saRNA targeting C/EBPα and at least one additional active agent. Methods of using the pharmaceutical composition are also provided.

### CROSS-REFERENCE TO RELATED APPLICATIONS

This application claims priority to U.S. Provisional Application No. 62/879,028, filed Jul. 26, 2019, entitled COMPOSITIONS AND METHODS OF USING C/EBP ALPHA SARNA, and U.S. Provisional Application No. 63/050,091, filed Jul. 9, 2020, entitled COMPOSITIONS AND METHODS OF USING C/EBP ALPHA SARNA, the contents of which are incorporated herein by reference in its entirety.

### REFERENCE TO SEQUENCE LISTING

The present application is being filed along with a Sequence Listing in electronic format. The sequence listing filed in ASCII format, entitled 2058-1027PCT_SL.txt, was created on Jul. 27, 2020 and is 6,725 bytes in size. The information in electronic format of the Sequence Listing is incorporated herein by reference in its entirety.

### FIELD OF THE DISCLOSURE

The disclosure relates to polynucleotide, specifically saRNA, compositions for modulating C/EBPα and C/EBPα pathways and to the methods of using the compositions in therapeutic applications.

### BACKGROUND

Myeloid-derived suppressor cells (MDSC) are closely related to neutrophils and monocytes. MDSC consist of two large groups of cells termed granulocytic or polymorphonuclear (PMN-MDSC), which are phenotypically and morphologically similar to neutrophils; and monocytic (M-MDSC)—phenotypically and morphologically similar to monocytes. MDSC cells have been discovered as an important contributor to tumor progression. They play a key role in immune suppression in cancer, as well as tumor angiogenesis, drug resistance, and promotion of tumor metastases.

Tumor immune suppression is an important feature of MDSC. MDSC is involved in suppression of different cells of the immune system, T cells being the major target. The main factors implicated in MDSC-mediated immune suppression include arginase (ARG1), iNOS, TGFβ, IL-10, COX2, indoleamine 2,3-dioxygenase (IDO) sequestration of cysteine, decrease of L-selectin expression by T-cells and many others. In additional to tumor immune suppression, MDSC also promotes tumor progression by affecting the tumor microenvironment and tumor angiogenesis. Thus, there is a need for regulating MDSC and inhibiting immune suppression involving MDSC.

### SUMMARY OF THE DISCLOSURE

The present disclosure provides a method of blocking MDSC's or TAM's inhibitory activity against T-cell proliferation in a subject in need thereof, comprising administering a synthetic isolated saRNA to the subject. The saRNA may comprise an antisense strand with a sequence of SEQ ID No. 1 (CEBPA-51).

The present disclosure also provides a method of up-regulating the expression of the C/EBPα gene in a cell in a subject in need thereof, wherein the cell is a monocytic myeloid-derived suppressor cell (MDSC) or a tumor associated macrophage (TAM), comprising administering a synthetic isolated saRNA to the cell. The saRNA may comprise an antisense strand with a sequence of SEQ ID No. 1 (CEBPA-51).

The present disclosure also provides a method of reducing the expression of a target gene in a cell in a subject in need thereof, comprising administering a synthetic isolated saRNA to the cell. The saRNA may comprise an antisense strand with a sequence of SEQ ID No. 1 (CEBPA-51), wherein the target gene is ARG1, iNOS, S100A8 or S100A9.

The present disclosure also provides a method of delivering a synthetic isolated saRNA to myeloid cells of a subject in need thereof, comprising formulating the saRNA with liposomes. The saRNA may comprise an antisense strand with a sequence of SEQ ID No. 1 (CEBPA-51).

The present disclosure also provides a method of treating cancer in a subject in need thereof, comprising administering a synthetic isolated saRNA to the cell, wherein the subject further receives a CTLA-4 inhibitor, a COX2 inhibitor, a FATP2 inhibitor, or a combination thereof. The saRNA may comprise an antisense strand with a sequence of SEQ ID No. 1 (CEBPA-51).

The details of various embodiments of the disclosure are set forth in the description below. Other features, objects, and advantages of the disclosure will be apparent from the description and the drawings, and from the claims.

### BRIEF DESCRIPTION OF THE DRAWINGS

FIG. 1 shows tumor areas after MTL-CEBPA treatment in the LLC tumor model.

FIG. 2A shows CEBPA expressions after MTL-CEBPA treatment.

FIG. 2B shows Arg1 and iNOS gene expressions after MTL-CEBPA treatment.

FIG. 3A shows % T cell proliferation changes by M-MDSC cells after MTL-CEBPA treatment compared with M-MDSC cells without MTL-CEBPA treatment.

FIG. 3B shows % T cell proliferation changes by TAM cells after MTL-CEBPA treatment compared with M-MDSC cells without MTL-CEBPA treatment.

FIG. 4 is a summary of the study with MC38 tumor model.

FIG. 5A shows tumor areas after MTL-CEBPA treatment in the MC38 tumor model.

FIG. 5B shows T cell proliferation changes by M-MDSC cells.

FIG. 5C shows T cell proliferation changes by TAM cells.

FIG. 6 is a summary of the study in Example 3.

FIG. 7A shows tumor areas after single agent treatments (MTL-CEBPA or CTLA4 Ab) and combo treatment with MTL-CEBPA+CTLA4 Ab in the LLC tumor model.

FIG. 7B shows tumor areas after single agent treatments (MTL-CEBPA or Celecoxib) and combo treatment with MTL-CEBPA+Celecoxib in the LLC tumor model.

FIG. 8 is a summary of the study in Example 4.

FIG. 9 shows tumor growth after single agent treatments (MTL-CEBPA or Lipofermata) and combo treatment with MTL-CEBPA+Lipofermata in the LLC tumor model.

### DETAILED DESCRIPTION

The present disclosure provides compositions and kits for nucleic acid constructs that target a C/EBPα transcript and methods of using these compositions and kits.

CCAAT/enhancer-binding protein α (C/EBPα, C/EBP alpha, C/EBPA or CEBPA) is a leucine zipper protein that is conserved across humans and rats. This nuclear transcription factor is enriched in hepatocytes, myelomonocytes, adipocytes, as well as other types of mammary epithelial cells [Lekstrom-Himes et al., J. Bio. Chem, vol. 273, 28545-28548 (1998)]. C/EBPα protein is known as a critical regulator of metabolic processes and cell proliferation. Modulating C/EBPα gene has great potentials for therapeutic purposes. The present disclosure provides nucleic acid constructs targeting a C/EBPα transcript, wherein the nucleic acid constructs may include single or double stranded DNA or RNA with or without modifications.

C/EBPα gene as used herein is a double-stranded DNA comprising a coding strand and a template strand. It may also be referred to the target gene in the present application.

The terms “C/EBPα transcript”, “C/EBPα target transcript” or “target transcript” in the context may be C/EBPα mRNA encoding C/EBPα protein. C/EBPα mRNA is transcribed from the template strand of C/EBPα gene and may exist in the mitochondria.

The antisense RNA of the C/EBPα gene transcribed from the coding strand of the C/EBPα gene is called a target antisense RNA transcript herein after. The target antisense RNA transcript may be a long non-coding antisense RNA transcript.

The terms “small activating RNA”, “short activating RNA”, or “saRNA” in the context of the present disclosure means a single-stranded or double-stranded RNA that upregulates or has a positive effect on the expression of a specific gene. The saRNA may be single-stranded of 14 to 30 nucleotides. The saRNA may also be double-stranded, each strand comprising 14 to 30 nucleotides. The gene is called the target gene of the saRNA. A saRNA that upregulates the expression of the C/EBPα gene is called a “C/EBPα-saRNA” and the C/EBPα gene is the target gene of the C/EBPα-saRNA.

The terms “target” or “targeting” in the context mean having an effect on a C/EBPα gene. The effect may be direct or indirect. Direct effect may be caused by complete or partial hybridization with the C/EBPα target antisense RNA transcript. Indirect effect may be upstream or downstream.

C/EBPα-saRNA may have a downstream effect on a biological process or activity. In such embodiments, C/EBPα-saRNA may have an effect (either upregulating or downregulating) on a second, non-target transcript.

The term “gene expression” in the context may include the transcription step of generating C/EBPα mRNA from C/EBPα gene or the translation step generating C/EBPα protein from C/EBPα mRNA. An increase of C/EBPα mRNA and an increase of C/EBPα protein both indicate an increase or a positive effect of C/EBPα gene expression.

By “upregulation” or “activation” of a gene is meant an increase in the level of expression of a gene, or levels of the polypeptide(s) encoded by a gene or the activity thereof, or levels of the RNA transcript(s) transcribed from the template strand of a gene above that observed in the absence of the saRNA of the present disclosure. By “downregulation” or “inhibition” of a gene is meant a decrease in the level of expression of a gene, or levels of the polypeptide(s) encoded by a gene or the activity thereof, or levels of the RNA transcript(s) transcribed from the template strand of a gene above that observed in the absence of the saRNA of the present disclosure. The saRNA of the present disclosure may have a direct or indirect upregulating or downregulating effect on the expression of a target gene.

In one embodiment, the saRNA of the present disclosure may show efficacy in proliferating cells. As used herein with respect to cells, “proliferating” means cells which are growing and/or reproducing rapidly.

### I. Composition of the Disclosure

One aspect of the present disclosure provides pharmaceutical compositions comprising a saRNA that upregulates CEBPA gene, and at least one pharmaceutically acceptable carrier. Such a saRNA is referred herein after as “C/EBPα-saRNA”, or “saRNA of the present disclosure”, used interchangeably in this application.

The C/EBPα-saRNA has 14-30 nucleotides and comprises a sequence that is at least 80%, 90%, 95%, 98%, 99% or 100% complementary to a targeted sequence on the template strand of the C/EBPα gene. The targeted sequence may have the same length, i.e., the same number of nucleotides, as the saRNA and/or the reverse complement of the saRNA. The relationships among the saRNAs, a target gene, a coding strand of the target gene, a template strand of the target gene, a targeted sequence/target site, and the transcription start site (TSS) are shown in FIG. 1.

In some embodiments, the targeted sequence comprises at least 14 and less than 30 nucleotides.

In some embodiments, the targeted sequence has 19, 20, 21, 22, or 23 nucleotides.

In some embodiments, the location of the targeted sequence is situated within a promoter area of the template strand.

In some embodiments, the targeted sequence of the C/EBPα-saRNA is located within a TSS (transcription start site) core of the template stand of the C/EBPα gene. A “TSS core” or “TSS core sequence” as used herein, refers to a region between 2000 nucleotides upstream and 2000 nucleotides downstream of the TSS (transcription start site). Therefore, the TSS core comprises 4001 nucleotides and the TSS is located at position 2001 from the 5′ end of the TSS core sequence. CEBPA TSS core sequence is show in the table below:

| CEBPA mRNA | CEBPA protein | CEBPA TSS core | CEBPA TSS core |
|---|---|---|---|
| REF. No. | REF. No. | genomic location | sequence ID No. |
| NM_001285829 | NP_001272758 | chr19:33302564 | SEQ ID No. 3 |
| NM_001287424 | NP_001274353 | minus strand | |
| NM_001287435 | NP_001274364 | | |
| NM_004364 | NP_004355 | | |

In some embodiments, the targeted sequence is located between 1000 nucleotides upstream and 1000 nucleotides downstream of the TSS.

In some embodiments, the targeted sequence is located between 500 nucleotides upstream and 500 nucleotides downstream of the TSS.

In some embodiments, the targeted sequence is located between 250 nucleotides upstream and 250 nucleotides downstream of the TSS.

In some embodiments, the targeted sequence is located between 100 nucleotides upstream and 100 nucleotides downstream of the TSS.

In some embodiments, the targeted sequence is located upstream of the TSS in the TSS core. The targeted sequence may be less than 2000, less than 1000, less than 500, less than 250, or less than 100 nucleotides upstream of the TSS.

In some embodiments, the targeted sequence is located downstream of the TSS in the TSS core. The targeted sequence may be less than 2000, less than 1000, less than 500, less than 250, or less than 100 nucleotides downstream of the TSS.

In some embodiments, the targeted sequence is located +/−50 nucleotides surrounding the TSS of the TSS core. In some embodiments, the targeted sequence substantially overlaps the TSS of the TSS core. In some embodiments, the targeted sequence begins or ends at the TSS of the TSS core. In some embodiments, the targeted sequence overlaps the TSS of the TSS core by 1, 2, 3, 4, 5, 6, 7, 8, 9, 10, 11, 12, 13, 14, 15, 16, 17, 18 or 19 nucleotides in either the upstream or downstream direction.

The location of the targeted sequence on the template strand is defined by the location of the 5′ end of the targeted sequence. The 5′ end of the targeted sequence may be at any position of the TSS core and the targeted sequence may start at any position selected from position 1 to position 4001 of the TSS core. For reference herein, when the 5′ most end of the targeted sequence from position 1 to position 2000 of the TSS core, the targeted sequence is considered upstream of the TSS and when the 5′ most end of the targeted sequence is from position 2002 to 4001, the targeted sequence is considered downstream of the TSS. When the 5′ most end of the targeted sequence is at nucleotide 2001, the targeted sequence is considered to be a TSS centric sequence and is neither upstream nor downstream of the TSS.

For further reference, for example, when the 5′ end of the targeted sequence is at position 1600 of the TSS core, i.e., it is the 1600^{th} nucleotide of the TSS core, the targeted sequence starts at position 1600 of the TSS core and is considered to be upstream of the TSS.

In one embodiment, the saRNA of the present disclosure may have two strands that form a duplex, one strand being a guide strand. The saRNA duplex is also called a double-stranded saRNA. A double-stranded saRNA or saRNA duplex, as used herein, is a saRNA that includes more than one, and preferably, two, strands in which interstrand hybridization can form a region of duplex structure. The two strands of a double-stranded saRNA are referred to as an antisense strand or a guide strand, and a sense strand or a passenger strand.

In some embodiments, the C/EBPα-saRNA may comprising any C/EBPα-saRNA disclosed in WO2015/075557 or WO2016/170349 to MiNA Therapeutics Limited, the contents of each of which are incorporated herein by reference in their entirety, such as saRNAs in Table 1, Table 1A, Table 3-1 and Table 3-2, AW51, and CEBPA-51 disclosed in WO2016/170349.

In some embodiments, the C/EBPα-saRNA may be modified and may comprising any modification disclosed in WO2016/170349 to MiNA Therapeutics Limited.

In one embodiment, the C/EBPα-saRNA is CEBPA-51 (or CEBPA51), which is an saRNA duplex that upregulates C/EBPα. Its design, sequences, and compositions/formulations are disclosed in the Detailed Description and Examples of WO2016/170349 to MiNA Therapeutics Limited. The sequences of the sense and antisense strands of CEBPA-51 are shown in Table 1.

TABLE 1
CEBPA-51 (CEBPA51) Sequences

| | | |
|---|---|---|
| Antisense | GACCAGUGACAAUGACCGCmUmU | SEQ ID No. 1 |
| Sense | (invabasic)mGmCGmGUCAUUmGUCAmCUGGUCmUmU | SEQ ID No. 2 |

mU, mG, and mC mean 2′-O-methyl modified U, G, and C.
invabasic = inverted abasic sugar cap.

The alignment of the strands is shown in the Table 2.

TABLE 2
CEBPA-51 Alignment of Strands

| | saRNA name | | | | | | |
|---|---|---|---|---|---|---|---|
| | CEBPA-51 Total base: 21 mer including base modifications | | | | | | |
| | mer | | | | | | |
| | 3 | 6 | 9 | 12 | 15 | 18 | 21 |
| Sense strand | bmGmCG | mGUC | AUU | mGUC | AmCU | GGU | CmUmU |
| 5′ → 3′ | | | | | | | |
| (SEQ ID No. 2) | | | | | | | |
| Complementary | mUmUC | GCC | AGU | AAC | AGU | GAC | CAG |
| antisense strand | | | | | | | |
| 3′ → 5′ | | | | | | | |
| (SEQ ID No. 1) | | | | | | | |

Definition of symbols: A, U, G, C are 2′-OH ribonucleotides, mU, mG, mC are 2′-O-methyl ribonucleotides,
b = inverted abasic sugar cap.

CEBPA-51 is encapsulated into liposomes (NOV340 SMARTICLES® technology owned by Marina Biotech) to make MTL-CEBPA. The lipid components of the NOV340 SMARTICLES® are comprised of 1-palmitoyl-2-oleoyl-sn-glycero-3-phosphocholine (POPC), 1,2-dioleoyl-sn-glycero-3-phosphoethanolamine (DOPE), cholesteryl-hemi succinate (CHEMS), and 4-(2-aminoethyl)-morpholino-cholesterol hemi succinate (MOCHOL). NOV340 SMARTICLES® consists of POPC, DOPE, CHEMS and MOCHOL in the molar ratio of 6:24:23:47. These nanoparticles are anionic at physiological pH, and their specific lipid ratio imparts a “pH-tunable” character and a charge to the liposomes, which changes depending upon the surrounding pH of the microenvironment to facilitate movement across physiologic membranes. SMARTICLES® nanoparticles are sized to avoid extensive immediate hepatic sequestration, with an average diameter of approximately about 50-about 150 nm, or about 100-about 120 nm, facilitating more prolonged systemic distribution and improved serum stability after i.v. injection leading to broader tissue distribution with high levels in liver, spleen and bone marrow reported.

MTL-CEBPA also comprises the buffer forming excipients such as sucrose and phosphate-salts. Qualitative and quantitative composition of MTL-CEBPA (2.5 mg/ml) are shown in Table 3.

Table 3
MTL-CEBPA Composition

| | | | Quantity |
|---|---|---|---|
| Name of Ingredient | Function | Reference | (per ml) |
| CEBPA-51 (saRNA) | Active | Manufacturer’s | 2.5 mg/ml |
| | pharmaceutical | specifications | |
| | ingredient | | |
| 1-palmitoyl-2-oleoyl-sn- | Membrane | Manufacturer’s | 4.65 mg/ml |
| glycero-3-phosphocholine (POPC) | forming lipid | specifications | |
| l,2-dioleoyl-sn-glycero-3- | Membrane forming | Manufacturer’s | 18.0 mg/ml |
| phosphoethanolamine (DOPE) | fusogenic lipid | specifications | |
| Cholesteryl hemisuccinate (CHEMS) | Anionic | Manufacturer’s | 11.3 mg/ml |
| | ampotheric lipid | specifications | |
| Cholesteryl-4-[[2-(4- | Cationic | Manufacturer’s | 27.0 mg/ml |
| morpholinyl)ethyl]amino]- | amphoteric lipid | specifications | |
| 4-oxobutanoate (MOCHOL) | | | |
| Sucrose | Cryoprotectant, | BP, JP, NF, EP | 92.4 mg/ml |
| | osmolality control | | |
| Disodium hydrogen phosphate, dihydrate | Buffer pH adjustment | BP, USP, EP | 1.44 mg/ml |
| Potassium dihydrogen phosphate | Buffer pH adjustment | EP, BP, NF | 0.2 mg/ml |
| Potassium chloride (KCl) | Ionic strength adjuster | EP, BP, USP | 0.2 mg/ml |
| Water for injection (WFI) | Solvent | WFI (USP, EP) | qs 1 ml |

Administration

C/EBPα-saRNAs or C/EBPα-saRNA compositions, such as CEBPA-51 and/or MTL-CEBPA, may be administered by any route which results in a therapeutically effective outcome. These include, but are not limited to enteral, gastroenteral, epidural, oral, transdermal, epidural (peridural), intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection, (into the base of the penis), intravaginal administration, intrauterine, extra-amniotic administration, transdermal (diffusion through the intact skin for systemic distribution), transmucosal (diffusion through a mucous membrane), insufflation (snorting), sublingual, sublabial, enema, eye drops (onto the conjunctiva), or in ear drops. In specific embodiments, compositions may be administered in a way which allows them to cross the blood-brain barrier, vascular barrier, or other epithelial barrier. Routes of administration disclosed in International Publication WO 2013/090648 filed Dec. 14, 2012, the contents of which are incorporated herein by reference in their entirety, may be used to administer the saRNA of the present disclosure.

Dosing

In some embodiments, C/EBPα-saRNAs or C/EBPα-saRNA compositions, such as CEBPA-51 and/or MTL-CEBPA, are administered once every day, once every 2 days, once every 3 days, once every 4 days, or once every 5 days.

In some embodiments, at least two doses of C/EBPα-saRNAs or C/EBPα-saRNA compositions, such as CEBPA-51 and/or MTL-CEBPA, are administered to a subject. The subject may have a liver disease, such as liver cancer, non-alcoholic steatohepatitis (NASH), steatosis, liver damage, liver failure, or liver fibrosis. The doses are less than 7 days apart. In one embodiment, CEBPA-51 and/or MTL-CEBPA is administered every 24 hours. In one embodiment, CEBPA-51 and/or MTL-CEBPA is administered every 48 hours.

In some embodiments, the patient receives at least 2 doses, e.g, 3 doses, 4 doses, 5 doses, 6 doses, 7 doses, 8 doses, 9 doses, or 10 doses, of C/EBPα-saRNAs or C/EBPα-saRNA compositions, such as CEBPA-51 and/or MTL-CEBPA.

In some embodiments, C/EBPα-saRNAs or C/EBPα-saRNA compositions, such as CEBPA-51 and/or MTL-CEBPA, are administered for a period of at least 2 days, such as 3 days, 4 days, 5 days, 6 days, 1 week, 8 days, 9 days, 10 days, 2 weeks, 3 weeks, 4 weeks, 5 weeks, or 6 weeks.

In one embodiment, CEBPA-51 and/or MTL-CEBPA is administered every 24 hours for a period of at least 2 days, such as 3 days, 4 days, 5 days, 6 days, 1 week, 8 days, 9 days, 10 days, 2 weeks, 3 weeks, 4 weeks, 5 weeks, or 6 weeks.

In one embodiment, CEBPA-51 and/or MTL-CEBPA is administered every 48 hours for a period of at least 2 days, such as 3 days, 4 days, 5 days, 6 days, 1 week, 8 days, 9 days, 10 days, 2 weeks, 3 weeks, 4 weeks, 5 weeks, or 6 weeks.

In some embodiments, C/EBPα-saRNAs or C/EBPα-saRNA compositions, such as CEBPA-51 and/or MTL-CEBPA, are administered via intravenous infusion over 60 minutes. Doses are between about 20 to about 160 mg/m².

The dosing regimen disclosed in the present application may apply to any indication or disorder that can be treated with C/EBPα-saRNAs or C/EBPα-saRNA compositions.

### II. Methods of Use

One aspect of the present disclosure provides methods of using C/EBPα-saRNA and pharmaceutical compositions comprising said C/EBPα-saRNA and at least one pharmaceutically acceptable carrier. C/EBPα-saRNA modulates C/EBPα gene expression. In one embodiment, the expression of C/EBPα gene is increased by at least 20, 30, 40%, more preferably at least 45, 50, 55, 60, 65, 70, 75%, even more preferably at least 80% in the presence of the saRNA of the present disclosure compared to the expression of C/EBPα gene in the absence of the saRNA of the present disclosure. In a further preferable embodiment, the expression of C/EBPα gene is increased by a factor of at least 2, 3, 4, 5, 6, 7, 8, 9, 10, more preferably by a factor of at least 15, 20, 25, 30, 35, 40, 45, 50, even more preferably by a factor of at least 60, 70, 80, 90, 100, in the presence of the saRNA of the present disclosure compared to the expression of C/EBPα gene in the absence of the saRNA of the present disclosure.

In one embodiment, the increase in gene expression of the saRNA descried herein is shown in proliferating cells.

### Hyperproliferation Disorders

In one embodiment of the disclosure, C/EBPα-saRNA of the present disclosure is used to reduce cell proliferation of hyperproliferative cells. Examples of hyperproliferative cells include cancerous cells, e.g., carcinomas, sarcomas, lymphomas and blastomas. Such cancerous cells may be benign or malignant. Hyperproliferative cells may result from an autoimmune condition such as rheumatoid arthritis, inflammatory bowel disease, or psoriasis. Hyperproliferative cells may also result within patients with an oversensitive immune system coming into contact with an allergen. Such conditions involving an oversensitive immune system include, but are not limited to, asthma, allergic rhinitis, eczema, and allergic reactions, such as allergic anaphylaxis. In one embodiment, tumor cell development and/or growth is inhibited. In a preferred embodiment, solid tumor cell proliferation is inhibited. In another preferred embodiment, metastasis of tumor cells is prevented. In another preferred example, undifferentiated tumor cell proliferation is inhibited.

Inhibition of cell proliferation or reducing proliferation means that proliferation is reduced or stops altogether. Thus, “reducing proliferation” is an embodiment of “inhibiting proliferation”. Proliferation of a cell is reduced by at least 20%, 30% or 40%, or preferably at least 45, 50, 55, 60, 65, 70 or 75%, even more preferably at least 80, 90 or 95% in the presence of the saRNA of the disclosure compared to the proliferation of said cell prior to treatment with the saRNA of the disclosure, or compared to the proliferation of an equivalent untreated cell. In embodiments wherein cell proliferation is inhibited in hyperproliferative cells, the “equivalent” cell is also a hyperproliferative cell. In preferred embodiments, proliferation is reduced to a rate comparable to the proliferative rate of the equivalent healthy (non-hyperproliferative) cell. Alternatively viewed, a preferred embodiment of “inhibiting cell proliferation” is the inhibition of hyperproliferation or modulating cell proliferation to reach a normal, healthy level of proliferation.

In one non-limiting example, C/EBPα-saRNA is used to reduce the proliferation of leukemia and lymphoma cells. Preferably, the cells include Jurkat cells (acute T cell lymphoma cell line), K562 cells (erythroleukemia cell line), U373 cells (glioblastoma cell line), and 32Dp210 cells (myeloid leukemia cell line).

In another non-limiting example, C/EBPα-saRNA is used to reduce the proliferation of ovarian cancer cells, liver cancer cells, pancreatic cancer cells, breast cancer cells, prostate cancer cells, rat liver cancer cells, and insulinoma cells. Preferably, the cells include PEO1 and PEO4 (ovarian cancer cell line), HepG2 (hepatocellular carcinoma cell line), Panc1 (human pancreatic carcinoma cell line), MCF7 (human breast adenocarcinoma cell line), DU145 (human metastatic prostate cancer cell line), rat liver cancer cells, and MING (rat insulinoma cell line).

In another non-limiting example, C/EBPα-saRNA is used in combination with a siRNA targeting C/EBPβ gene to reduce tumor cell proliferation. Tumor cell may include hepatocellular carcinoma cells such as HepG2 cells and breast cancer cells such as MCF7 cells.

In one embodiment, the saRNA of the present disclosure is used to treat hyperproliferative disorders. Tumors and cancers represent a hyperproliferative disorder of particular interest, and all types of tumors and cancers, e.g. solid tumors and haematological cancers are included. Examples of cancer include, but not limited to, cervical cancer, uterine cancer, ovarian cancer, kidney cancer, gallbladder cancer, liver cancer, head and neck cancer, squamous cell carcinoma, gastrointestinal cancer, breast cancer, prostate cancer, testicular cancer, lung cancer, non-small cell lung cancer, non-Hodgkin's lymphoma, multiple myeloma, leukemia (such as acute lymphocytic leukemia, chronic lymphocytic leukemia, acute myelogenous leukemia, and chronic myelogenous leukemia), brain cancer (e.g. astrocytoma, glioblastoma, medulloblastoma), neuroblastoma, sarcomas, colon cancer, rectum cancer, stomach cancer, anal cancer, bladder cancer, endometrial cancer, plasmacytoma, lymphomas, retinoblastoma, Wilm's tumor, Ewing sarcoma, melanoma and other skin cancers. The liver cancer may include, but not limited to, cholangiocarcinoma, hepatoblastoma, haemangiosarcoma, or hepatocellular carcinoma (HCC). HCC is of particular interest.

Primary liver cancer is the fifth most frequent cancer worldwide and the third most common cause of cancer-related mortality. HCC represents the vast majority of primary liver cancers [El-Serag et al., Gastroenterology, vol. 132(7), 2557-2576 (2007), the contents of which are disclosed herein in their entirety]. HCC is influenced by the interaction of several factors involving cancer cell biology, immune system, and different aetiologies (viral, toxic and generic). The majority of patients with HCC develop malignant tumors from a background of liver cirrhosis. Currently most patients are diagnosed at an advanced stage and therefore the 5 year survival for the majority of HCC patients remains dismal. Surgical resection, loco-regional ablation and liver transplantation are currently the only therapeutic options which have the potential to cure HCC. However, based on the evaluation of individual liver function and tumor burden only about 5-15% of patients are eligible for surgical intervention. The binding sites for the family of C/EBP transcription factors are present in the promoter regions of numerous genes that are involved in the maintenance of normal hepatocyte function and response to injury (including albumin, interleukin 6 response, energy homeostasis, ornithine cycle regulation and serum amyloid A expression). The present disclosure utilizes C/EBPα-saRNA to modulate the expression of C/EBPα gene and treat liver cirrhosis and HCC.

The method of the present disclosure may reduce tumor volume by at least 10, 20, 30, 40, 50, 60, 70, 80 or 90%. Preferably, the development of one or more new tumors is inhibited, e.g. a subject treated according to the disclosure develops fewer and/or smaller tumors. Fewer tumors means that he develops a smaller number of tumors than an equivalent subject over a set period of time. For example, he develops at least 1, 2, 3, 4 or 5 fewer tumors than an equivalent control (untreated) subject. Smaller tumor means that the tumors are at least 10, 20, 30, 40, 50, 60, 70, 80 or 90% smaller in weight and/or volume than tumors of an equivalent subject. The method of the present disclosure reduces tumor burden by at least 10, 20, 30, 40, 50, 60, 70, 80 or 90%.

The set period of time may be any suitable period, e.g. 1, 2, 3, 4, 5, 6, 7, 8, 9 or 10 months or years.

In one non-limiting example, provided is a method of treating an undifferentiated tumor, comprising contacting a cell, tissue, organ or subject with C/EBPα-saRNA of the present disclosure. Undifferentiated tumors generally have a poorer prognosis compared to differentiated ones. As the degree of differentiation in tumors has a bearing on prognosis, it is hypothesized that the use of a differentiating biological agent could be a beneficial anti-proliferative drug. C/EBPα is known to restore myeloid differentiation and prevent hyperproliferation of hematopoietic cells in acute myeloid leukemia. Preferably, undifferentiated tumors that may be treated with C/EBPα-saRNA include undifferentiated small cell lung carcinomas, undifferentiated pancreatic adenocarcinomas, undifferentiated human pancreatic carcinoma, undifferentiated human metastatic prostate cancer, and undifferentiated human breast cancer.

In one non-limiting example, C/EBPα-saRNA is complexed into PAMAM dendrimer, referred to as C/EBPα-saRNA-dendrimer for targeted in vivo delivery. The therapeutic effect of intravenously injected C/EBPα-saRNA-dendrimers is demonstrated in a clinically relevant rat liver tumor model as shown in Example 1. After three doses through tail vein injection at 48 hour intervals, the treated cirrhotic rats showed significantly increased serum albumin levels within one week. The liver tumor burden was significantly decreased in the C/EBPα-saRNA dendrimer treated groups. This study demonstrates, for the first time, that gene targeting by small activating RNA molecules can be used by systemic intravenous administration to simultaneously ameliorate liver function and reduce tumor burden in cirrhotic rats with HCC.

In one embodiment, C/EBPα-saRNA is used to regulate oncogenes and tumor suppressor genes. Preferably, the expression of the oncogenes may be down-regulated. The expression of the oncogenes reduces by at least 20, 30, 40%, more preferably at least 45, 50, 55, 60, 65, 70, 75, 80, 85, 90, 95% in the presence of C/EBPα-saRNA of the disclosure compared to the expression in the absence of C/EBPα-saRNA of the disclosure. In a further preferable embodiment, the expression of the oncogenes is reduced by a factor of at least 2, 3, 4, 5, 6, 7, 8, 9, 10, more preferably by a factor of at least 15, 20, 25, 30, 35, 40, 45, 50, even more preferably by a factor of at least 60, 70, 80, 90, 100, in the presence of C/EBPα-saRNA of the disclosure compared to the expression in the absence of C/EBPα-saRNA of the disclosure. Preferably, the expressions of tumor suppressor genes may be inhibited. The expression of the tumor suppressor genes increase by at least 20, 30, 40%, more preferably at least 45, 50, 55, 60, 65, 70, 75, 80, 85, 90, 95%, even more preferably at least 100% in the presence of C/EBPα-saRNA of the disclosure compared to the expression in the absence of C/EBPα-saRNA of the disclosure. In a further preferable embodiment, the expression of tumor suppressor genes is increased by a factor of at least 2, 3, 4, 5, 6, 7, 8, 9, 10, more preferably by a factor of at least 15, 20, 25, 30, 35, 40, 45, 50, even more preferably by a factor of at least 60, 70, 80, 90, 100 in the presence of C/EBPα-saRNA of the disclosure compared to the expression in the absence of C/EBPα-saRNA of the disclosure. Non-limiting examples of oncogenes and tumor suppressor genes include Bcl-2-associated X protein (BAX), BH3 interacting domain death agonist (BID), caspase 8 (CASP8), disabled homolog 2-interacting protein (DAB21P), deleted in liver cancer 1 (DLC1), Fas surface death receptor (FAS), fragile histidine triad (FHIT), growth arrest and DNA-damage-inducible-beta (GADD45B), hedgehog interacting protein (HHIP), insulin-like growth factor 2 (IGF2), lymphoid enhancer-binding factor 1 (LEF1), phosphatase and tensin homolog (PTEN), protein tyrosine kinase 2 (PTK2), retinoblastoma 1 (RB1), runt-related transcription factor 3 (RUNX3), SMAD family member 4 (SMAD4), suppressor of cytokine signaling (3 SOCS3), transforming growth factor, beta receptor II (TGFBR2), tumor necrosis factor (ligand) superfamily, member 10 (TNFSF10), P53, disintegrin and metalloproteinase domain-containing protein 17(ADAM17), v-akt murine thymoma viral oncogene homolog 1 (AKT1), angiopoietin 2 (ANGPT2), B-cell CLL/lymphoma 2 (BCL2), BCL2-like 1 (BCL2L1), baculoviral IAP repeat containing 2 (BIRC2), baculoviral IAP repeat containing 5 (BIRC5), chemokine (C-C motif) ligand 5 (CCL5), cyclin D1 (CCND1), cyclin D2 (CCND2), cadherin 1 (CDH1), cadherin 13 (CDH13), cyclin-dependent kinase inhibitor 1A (CDKN1A), cyclin-dependent kinase inhibitor 1B (CDKN1B), cyclin-dependent kinase inhibitor 2A (CDKN2A), CASP8 and FADD-like apoptosis regulator (CFLAR), catenin (cadherin-associated protein) beta 1 (CTNNB1), chemokine receptor 4 (CXCR4), E2F transcription factor 1 (E2F1), epidermal growth factor (EGF), epidermal growth factor receptor (EGFR), E1A binding protein p300 (EP300), Fas (TNFRSF6)-associated via death domain (FADD), fms-related tyrosine kinase 1 (FLT1), frizzled family receptor 7 (FZD7), glutathione S-transferase pi 1 (GSTP1), hepatocyte growth factor (HGF), Harvey rat sarcoma viral oncogene homolog (HRAS), insulin-like growth factor binding protein 1 (IGFBP1), insulin-like growth factor binding protein 3 (IGFBP3), insulin receptor substrate 1 (IRS1), integrin beta 1 (ITGB1), kinase insert domain receptor (KDR), myeloid cell leukemia sequence 1 (MCL1), met proto-oncogene (MET), mutS homolog 2 (MSH2), mutS homolog 3 (MSH3), metadherin (MTDH), v-myc avian myelocytomatosis viral oncogene homolog (MYC), nuclear factor of kappa light polypeptide gene enhancer in B-cells 1 (NFKB1), neuroblastoma RAS viral (v-ras) oncogene homolog (NRAS), opioid binding protein/cell adhesion molecule-like (OPCML), platelet-derived growth factor receptor, alpha polypeptide (PDGFRA), peptidylprolyl cis/trans isomerase, NIMA-interacting 1 (PIN1), prostaglandin-endoperoxide synthase 2 (PTGS2), PYD and CARD domain containing (PYCARD), ras-related C3 botulinum toxin substrate 1 (RAC1), Ras association (RalGDS/AF-6) domain family member 1 (RASSF1), reelin (RELN), ras homolog family member A (RHOA), secreted frizzled-related protein 2 (SFRP2), SMAD family member 7 (SMAD7), suppressor of cytokine signaling 1 (SOCS1), signal transducer and activator of transcription 3 (STAT3), transcription factor 4 (TCF4), telomerase reverse transcriptase (TERT), transforming growth factor alpha (TGFA), transforming growth factor beta 1 (TGFB1), toll-like receptor 4 (TLR4), tumor necrosis factor receptor superfamily member 10b (TNFRSF10B), vascular endothelial growth factor A (VEGFA), Wilms tumor 1 (WT1), X-linked inhibitor of apoptosis (XIAP), and Yes-associated protein 1 (YAP1).

In one embodiment, provided is a method of increasing white blood cell count by administering C/EBPα-saRNA of the present disclosure to a patient in need thereof. Also provided is a method of treating leukopaenia for patients having sepsis or chronic inflammation diseases (e.g., hepatitis and liver cirrhosis) and for immunocompromised patients (e.g., patients undergoing chemotherapy) by administering C/EBPα-saRNA of the present disclosure to said patient. Also provided is a method of treating pre B cell and B cell malignancies including leukaemia and lymphoma by administering C/EBPα-saRNA of the present disclosure to a patient in need thereof. Also provided is a method of mobilize white blood cells, haematopoietic or mesenchymal stem cells by administering C/EBPα-saRNA of the present disclosure to a patient in need thereof. In one embodiment, the white blood cell count in a patient treated with C/EBPα-saRNA is increased by at least 50%, 75%, 100%, more preferably by at least a factor of 1.5, 2, 2.5, 3, 3.5, 4, 4.5, 5, more preferably by at least a factor of 6, 7, 8, 9, 10 compared to no C/EBPα-saRNA treatment.

In one embodiment, C/EBPα-saRNA is used to regulate micro RNAs (miRNA or miR) in the treatment of hepatocellular carcinoma. MicroRNAs are small non-coding RNAs that regulate gene expression. They are implicated in important physiological functions and they may be involved in every single step of carcinogenesis. They typically have 21 nucleotides and regulate gene expression at the post transcriptional level via blockage of mRNA translation or induction of mRNA degradation by binding to the 3′-untranslated regions (3′-UTR) of said mRNA.

In tumors, regulation of miRNA expression affects tumor development. In HCC, as in other cancers, miRNAs function either as oncogenes or tumor suppressor genes influencing cell growth and proliferation, cell metabolism and differentiation, apoptosis, angiogenesis, metastasis and eventually prognosis. [Lin et al., Biochemical and Biophysical Research Communications, vol. 375, 315-320 (2008); Kutay et al., J. Cell. Biochem., vol. 99, 671-678 (2006); Meng et al., Gastroenterology, vol. 133(2), 647-658 (2007), the contents of each of which are incorporated herein by reference in their entirety] C/EBPα-saRNA of the present disclosure modulates C/EBPα gene expression and/or function and also regulates miRNA levels in HCC cells. Non-limiting examples of miRNAs that may be regulated by C/EBPα-saRNA of the present disclosure include hsa-let-7a-5p, hsa-miR-133b, hsa-miR-122-5p, hsa-miR-335-5p, hsa-miR-196a-5p, hsa-miR-142-5p, hsa-miR-96-5p, hsa-miR-184, hsa-miR-214-3p, hsa-miR-15a-5p, hsa-1et-7b-5p, hsa-miR-205-5p, hsa-miR-181a-5p, hsa-miR-140-5p, hsa-miR-146b-5p, hsa-miR-34c-5p, hsa-miR-134, hsa-1et-7g-5p, hsa-1et-7c, hsa-miR-218-5p, hsa-miR-206, hsa-miR-124-3p, hsa-miR-100-5p, hsa-miR-10b-5p, hsa-miR-155-5p, hsa-miR-1, hsa-miR-150-5p, hsa-1et-7i-5p, hsa-miR-27b-3p, hsa-miR-1et-7i-5p, hsa-miR-191-5p, hsa-1et-7f-5p, hsa-miR-10a-5p, hsa-miR-15b-5p, hsa-miR-16-5p, hsa-miR-34a-5p, hsa-miR-144-3p, hsa-miR-128, hsa-miR-215, hsa-miR-193a-5p, hsa-miR-23b-3p, hsa-miR-203a, hsa-miR-30c-5p, hsa-1et-7e-5p, hsa-miR-146a-5p, hsa-1et-7d-5p, hsa-miR-9-5p, hsa-miR-181b-5p, hsa-miR-181c-5p, hsa-miR-20b-5p, hsa-miR-125a-5p, hsa-miR-148b-3p, hsa-miR-92a-3p, hsa-miR-378a-3p, hsa-miR-130a-3p, hsa-miR-20a-5p, hsa-miR-132-3p, hsa-miR-193b-3p, hsa-miR-183-5p, hsa-miR-148a-3p, hsa-miR-138-5p, hsa-miR-373-3p, hsa-miR-29b-3p, hsa-miR-135b-5p, hsa-miR-21-5p, hsa-miR-181d, hsa-miR-301a-3p, hsa-miR-200c-3p, hsa-miR-7-5p, hsa-miR-29a-3p, hsa-miR-210, hsa-miR-17-5p, hsa-miR-98-5p, hsa-miR-25-3p, hsa-miR-143-3p, hsa-miR-19a-3p, hsa-miR-18a-5p, hsa-miR-125b-5p, hsa-miR-126-3p, hsa-miR-27a-3p, hsa-miR-372, hsa-miR-149-5p, and hsa-miR-32-5p.

In one non-limiting example, the miRNAs are oncogenic miRNAs and are downregulated by a factor of at least 0.01, 0.02, 0.05, 0.1, 0.2, 0.3, 0.5, 1, 1.5, 2, 2.5, and 3, in the presence of C/EBPα-saRNA of the disclosure compared to in the absence of C/EBPα-saRNA. In another non-limiting example, the miRNAs are tumor suppressing miRNAs and are upregulated by a factor of at least 0.01, 0.02, 0.05, 0.1, 0.2, 0.3, 0.5, 1, more preferably by a factor of at least 2, 3, 4, 5, 6, 7, 8, 9, 10, more preferably by a factor of at least 15, 20, 25, 30, 35, 40, 45, 50, even more preferably by a factor of at least 60, 70, 80, 90, 100, in the presence of C/EBPα-saRNA of the disclosure compared to in the absence of C/EBPα-saRNA.

### Regulating Immune Systems

Tumors are growing organs in the body initiated by oncogenic mutations and contains different population of immune cells. The prognostic outcome of a tumor is determined not only by the type of mutation of the tumor but also by the tumor stromal composition especially immune cells, In some embodiments, C/EBPα-saRNA of the present disclosure is used to regulate the immune system and/or immune cells of a subject.

In some embodiments, C/EBPα-saRNA of the present disclosure is used to regulate MDSC. In cancer and chronic inflammation, the bone marrow and spleen increase the generation of mature and immature myeloid cells that comprise a spectrum between monocytes and neutrophils. In mice, subpopulations of MDSCs, PMN-MDSC and M-MDSC, can be identified by exclusion of doublets, gating live CD11b+ cells, and evaluating the proportion of Ly6C and Ly6G cells. Mouse MDSC toward the neutrophil end of the spectrum (PMN-MDSC) shows a typical phenotype of CD11b⁺Ly6C^{lo}Ly6G⁺, while mouse MDSC toward the monocytic end of the spectrum (M-MDSC) shows a typical phenotype of CD11b⁺Ly6C^{hi}Ly6G⁻. Human PMN-MDSC shows a typical phenotype of CD14⁻CD11b⁻CD15⁺ (or CD66b⁺). Human M-MDSC shows a typical phenotype of CD11b⁺CD14⁺HLA-Dr^{low/-}CD15⁻.

Main features of MDSC include immune suppression and tumor-promoting activities. Although MDSCs were implicated in suppression of different cells of the immune system, T cells are the main targets of MDSCs. The immune regulatory activity of MDSCs depends on the metabolic consumption and conversion of the amino acids L-arginine and L-tryptophan, by the activity of inducible enzymes such as arginase 1 (ARG1), nitric oxide synthase 2 (NOS2/iNOS). Biomarkers associated with MDSC characterization include transcription factors and apoptotic regulators such as IRF8, phospho-STAT3, CEBP/β, S100A8/9, RB, phospho-STAT5, ROR/RORC1, sXBP, and CHOP; genes and molecules contributing to the immune-regulatory activity such as ARG1, NOS2/NO, NOX2/ROS, PNT/RNS, VEGF, PGE, and PD-L1; cytokines and receptors such as IL-10, TGFβ, and IL-4R (CD124), GM-CSF, G-CSF, IL-13 and IL-1.

In some embodiments, C/EBPα-saRNA of the present disclosure is used to regulate tumor associated macrophages (TAM). TAM has been shown to protect cancer cells from the anti-tumor immune responses and may be an important factor for tumor immune checkpoint mechanism. TAM expresses programmed cell death ligand 1 (PD-L1), PD-L2, CD80, and CD86 that restrict CD8+ T cell activities upon binding to the immune-checkpoint receptors, programmed cell death protein 1 (PD1) and cytotoxic T-lymphocyte-associated protein 4 (CTLA4). Macrophages isolated from the mouse and human tumors can directly suppress T cell responses in vitro, and that depletion of TAM enhances CD8+ T cell-mediated anti-tumor immunity in the mammary tumors in mice under treatment with chemotherapy.

Therefore, TAM are immune suppressor cells in tumors that restrict anti-tumor immune reaction induced by CD8+ T cells.

In some embodiments, C/EBPα-saRNA of the present disclosure is used to inhibit the immune suppression of MDSCs including M-MDSC and PMN-MDSC, or TAMs in a subject. For example, MDSC's or TAM's inhibitory activity against T-cell proliferation in a subject may be reduced by C/EBPα-saRNA of the present disclosure, such as a synthetic isolated saRNA comprising an antisense strand with a sequence of SEQ ID No. 1 (CEBPA-51). The saRNA may be double-stranded and further comprises a sense strand of SEQ ID No. 2 (CEBPA-51). CEBPA-51 may be delivered with liposomes such as NOV340 Smarticles. The T-cell proliferation may be up-regulated by at least 20%, 50%, 100%, 2 folds, 3 folds, 4 folds or 5 folds. The subject may have tumor, such as lung cancer or colon cancer.

In some embodiments, C/EBPα-saRNA of the present disclosure is used to regulate the expression of a target gene in an immune cell in a subject in need thereof, comprising administering C/EBPα-saRNA of the present disclosure to the cell. The immune cells may be MDSCs such as M-MDSC or PMN-MDSC, or TAMs. In some embodiments, the target gene may be C/EBPα, wherein the target gene expression is up-regulated by at least 20%, 50%, 100%, 2 folds, 3 folds, 4 folds or 5 folds by C/EBPα-saRNA of the present disclosure. In some embodiments, the target gene may be ARG1, iNOS, S100A8 or S100A9, wherein the target gene expression is reduced by at least 10%, 20%, 30%, 40%, 50%, 60%, 70%, or 80% by C/EBPα-saRNA of the present disclosure. The C/EBPα-saRNA of the present disclosure may be a synthetic isolated saRNA comprising an antisense strand with a sequence of SEQ ID No. 1 (CEBPA-51). The C/EBPα-saRNA of the present disclosure may be double-stranded and further comprises a sense strand of SEQ ID No. 2 (CEBPA-51). CEBPA-51 may be delivered with liposomes such as NOV340 Smarticles. The subject may have tumor, such as lung cancer or colon cancer.

Combination with Other Therapies

The saRNA of the present disclosure may be provided in combination with additional active agents or therapies known to have an effect in the particular method being considered. For example, the combination therapy comprising saRNA and additional active agents or therapies may be given to any patient in need thereof to treat any disorder described herein, including metabolics regulation, surgical care, hyperproliferative disorders, and/or stem cell regulation.

The additional active agents may be administered simultaneously or sequentially with the saRNA. The additional active agents may be administered in a mixture with the saRNA or be administered separately from the saRNA.

The term “administered simultaneously” as used herein is not specifically restricted and means that the components of the combination therapy, i.e., saRNA of the present disclosure and the additional active agents, are substantially administered at the same time, e.g. as a mixture or in immediate subsequent sequence.

The term “administered sequentially” as used herein is not specifically restricted and means that the components of the combination therapy, i.e., saRNA of the present disclosure and the additional active agents, are not administered at the same time but one after the other, or in groups, with a specific time interval between administrations. The time interval may be the same or different between the respective administrations of the components of the combination therapy and may be selected, for example, from the range of 2 minutes to 96 hours, 1 to 7 days or one, two or three weeks. Generally, the time interval between the administrations may be in the range of a few minutes to hours, such as in the range of 2 minutes to 72 hours, 30 minutes to 24 hours, or 1 to 12 hours. Further examples include time intervals in the range of 24 to 96 hours, 12 to 36 hours, 8 to 24 hours, and 6 to 12 hours. In some embodiments, the saRNA of the present disclosure is administered before the additional active agents. In some embodiments, the additional active agents are administered before the saRNA of the present disclosure.

The molar ratio of the saRNA of the present disclosure and the additional active agents is not particularly restricted. For example, when two components are combined in a composition, the molar ratio between the two components may be in the range of 1:500 to 500:1, or of 1:100 to 100:1, or of 1:50 to 50:1, or of 1:20 to 20:1, or of 1:5 to 5:1, or 1:1. Similar molar ratios apply when more than two components are combined in a composition. Each component may comprise, independently, a predetermined molar weight percentage from about 1% to 10%, or about 10% to about 20%, or about 20% to about 30%, or about 30% to 40%, or about 40% to 50%, or about 50% to 60%, or about 60% to 70%, or about 70% to 80%, or about 80% to 90%, or about 90% to 99% of the composition.

In one embodiment, C/EBPα-saRNA is administered with saRNA modulating a different target gene. Non-limiting examples include saRNA that modulates albumin, insulin or HNF4A genes. Modulating any gene may be achieved using a single saRNA or a combination of two or more different saRNAs. Non-limiting examples of saRNA that can be administered with C/EBPα-saRNA of the present disclosure include saRNA modulating albumin or HNF4A disclosed in International Publication WO 2012/175958 filed Jun. 20, 2012, saRNA modulating insulin disclosed in International Publications WO 2012/046084 and WO 2012/046085 both filed Oct. 10, 2011, saRNA modulating human progesterone receptor, human major vault protein (hMVP), E-cadherin gene, p53 gene, or PTEN gene disclosed in U.S. Pat. No. 7,709,456 filed Nov. 13, 2006 and US Pat. Publication US 2010/0273863 filed Apr. 23, 2010, and saRNAs targeting p21 gene disclosed in International Publication WO 2006/113246 filed Apr. 11, 2006, the contents of each of which are incorporated herein by reference in their entirety.

In one embodiment, C/EBPα-saRNA is administered in combination with a small interfering RNA or siRNA that inhibits the expression of C/EBPβ gene, i.e., C/EBPβ-siRNA.

In one embodiment, C/EBPα-saRNA is administered with one or more drugs that regulate metabolics, particularly liver function. In a non-limiting example, C/EBPα-saRNA of the present disclosure is administered with drugs that decrease low density lipoprotein (LDL) cholesterol levels, such as statin, simvastatin, atorvastatin, rosuvastatin, ezetimibe, niacin, PCSK9 inhibitors, CETP inhibitors, clofibrate, fenofibric, tocotrienols, phytosterols, bile acid sequestrants, probucol, or a combination thereof. C/EBPα-saRNA may also be administered with vanadium biguanide complexes disclosed in U.S. Pat. No. 6,287,586 to Orvig et al. In another example, C/EBPα-saRNA may be administered with a composition disclosed in WO 201102838 to Rhodes, the contents of which are incorporated by reference in their entirety, to lower serum cholesterol. The composition comprises an antigen binding protein that selectively binds to and inhibits a PCSK9 protein; and an RNA effector agent which inhibits the expression of a PCSK9 gene in a cell. In yet another example, C/EBPα-saRNA may be administered with an ABC1 polypeptide having ABC1 biological activity, or a nucleic acid encoding an ABC1 polypeptide having ABC1 activity to modulate cholesterol levels as described in EP1854880 to Brooks-Wilson et al., the contents of which are incorporated herein by reference in their entirety.

In another embodiment, C/EBPα-saRNA of the present disclosure is administered with drugs that increase insulin sensitivity or treat type II diabetes mellitus, such as metformin, sulfonylurea, nonsulfonylurea secretagogues, a glucosidase inhibitors, thiazolidinediones, pioglitazone, rosiglitazone, glucagon-like peptide-1 analog, and dipeptidyl peptidase-4 inhibitors or a combination thereof. Other hepato-protective agents that may be administered in combination with the saRNA of the present disclosure are disclosed in Adams et al., Postgraduate Medical Journal, vol. 82, 315-322 (2006), the contents of which are incorporated herein by reference in their entirety.

Immunotherapies

In some embodiments, the C/EBPα-saRNA and/or compositions of the present application may be combined with another therapy, such as surgical treatment, radiation therapy, immunotherapy, gene therapy, and/or with any other antineoplastic treatment method.

As used herein, the term “immunotherapy” refers to any therapy that can provoke and/or enhance an immune response to destroy tumor cells in a subject.

In some embodiments, the C/EBPα-saRNA and/or compositions of the present application may be combined with cancer vaccines and/or complementary immunotherapeutics such as immune checkpoint inhibitors. As used herein, the term “vaccine” refers to a composition for generating immunity for the prophylaxis and/or treatment of diseases.

In some embodiments, the checkpoint inhibitor may be an antagonist agent against CTLA-4 such as an antibody, a functional fragment of the antibody, a polypeptide, or a functional fragment of the polypeptide, or a peptide, which can bind to CTLA-4 with high affinity and prevent the interaction of B7-1/2 (CD80/86) with CTLA-4. In one example, the CTLA-4 antagonist is an antagonistic antibody, or a functional fragment thereof. Suitable anti-CTLA-4 antagonistic antibody include, without limitation, anti-CTLA-4 antibodies, human anti-CTLA-4 antibodies, mammalian anti-CTLA-4 antibodies, humanized anti-CTLA-4 antibodies, monoclonal anti-CTLA-4 antibodies, polyclonal anti-CTLA-4 antibodies, chimeric anti-CTLA-4 antibodies, MDX-010 (ipilimumab), tremelimumab (fully humanized), anti-CD28 antibodies, anti-CTLA-4 adnectins, anti-CTLA-4 domain antibodies, single chain anti-CTLA-4 antibody fragments, heavy chain anti-CTLA-4 fragments, light chain anti-CTLA-4 fragments, and the antibodies disclosed in U.S. Pat. Nos.: 8,748,815; 8,529,902; 8,318,916; 8,017,114; 7,744,875; 7,605,238; 7,465,446; 7,109,003; 7,132,281; 6,984,720; 6,682,736; 6,207,156; 5,977,318; and European Patent No. EP1212422B1; and U.S. Publication Nos. US 2002/0039581 and US 2002/086014; and Hurwitz et al., Proc. Natl. Acad. Sci. USA, 1998, 95(17):10067-10071; the contents of each of which are incorporated by reference herein in their entirety.

Additional anti-CTLA-4 antagonist agents include, but are not limited to, any inhibitors that are capable of disrupting the ability of CTLA-4 to bind to the ligands CD80/86.

In some embodiments, the checkpoint inhibitor may be agents used for blocking the PD-1 pathway include antagonistic peptides/antibodies and soluble PD-L1 ligands (See Table 4).

Table 4
Agents that block the inhibitory PD-1 and PD-L1 pathway

| Agent | Description | Target |
|---|---|---|
| Nivolumab (BMS-936558, | Human IgG | PD-1 |
| ONO-4538, MDX-1106 | | |
| Pembrolizumab | Humanized IgG4 | PD-1 |
| (MK-3475, lambrolizumab, | | |
| Keytruda ®) | | |
| Pidilizumab (CT-011) | Humanized anti- | PD-1 |
| | PD-1 IgG1kappa | |
| AMP-224 | B7-DC/IgG1 | PD-1 |
| | fusion protein | |
| MSB0010718 | Human IgG1 | PD-L1 |
| (EMD-Serono) | | |
| MEDI4736 | Engineered human | PD-L1 |
| | IgG1kappa | |
| MPDL3280A | Engineered IgG1 | PD-L1 |
| AUNP-12 | branched 29-amino | PD-1 |
| | acid peptide | |

In some embodiments, the C/EBPα-saRNA and/or compositions of the present application may be combined with a gene therapy, such as CRISPR (Clustered Regularly Interspaced Short Palidromic Repeats) therapy. As used herein, CRISPR therapy refers to any treatment that involves CRISPR-Cas system for gene editing.

In some embodiments, C/EBPα-saRNA of the present disclosure may be used in combination with one or more immune checkpoint blockade (ICB) agent. The combination may have synergistic effect on preventing and/or treating any cancer, such as but not limited to HCC.

In some embodiments, the ICB is a small inhibiting RNA (siRNA). The siRNA may be single stranded or double stranded.

In some embodiments, the ICB is an antibody.

In some embodiments, the ICB is a small molecule.

In some embodiments, the ICB is any agent in checkpoint inhibitor in Table 4.

In some embodiments, the ICB is Pembroluzimab, Tremelimumab, Durvalumab or Nivolumab.

In some embodiments, the patients receiving a combination therapy of C/EBPα-saRNA and at least one ICB may have HCC. The patients may be treated with an ICB first, followed by a treatment with C/EBPα-saRNA; be treated with C/EBPα-saRNA first, followed by a treatment with an ICB; or be treated with a composition comprising both C/EBPα-saRNA and ICB.

In some embodiments, the C/EBPα-saRNA and/or compositions of the present application may be combined with a CTLA-4 inhibitor. In some embodiments, the C/EBPα-saRNA and/or compositions of the present application may be combined with a COX2 inhibitor. In some embodiments, the C/EBPα-saRNA and/or compositions of the present application may be combined with a FATP2 inhibitor. In some embodiments, the C/EBPα-saRNA and/or compositions of the present application may be combined with at least one of a CTLA-4 inhibitor, a COX2 inhibitor, and a FATP2 inhibitor. In some embodiments, the C/EBPα-saRNA and/or compositions of the present application may be combined with at least two of a CTLA-4 inhibitor, a COX2 inhibitor, and a FATP2 inhibitor. In some embodiments, the C/EBPα-saRNA and/or compositions of the present application may be combined with a CTLA-4 inhibitor, a COX2 inhibitor, and a FATP2 inhibitor.

### III. Kits and Devices

### Kits

The disclosure provides a variety of kits for conveniently and/or effectively carrying out methods of the present disclosure. Typically, kits will comprise sufficient amounts and/or numbers of components to allow a user to perform multiple treatments of a subject(s) and/or to perform multiple experiments.

In one embodiment, the kits comprising saRNA described herein may be used with proliferating cells to show efficacy.

In one embodiment, the present disclosure provides kits for regulate the expression of genes in vitro or in vivo, comprising C/EBPα-saRNA of the present disclosure or a combination of C/EBPα-saRNA, saRNA modulating other genes, siRNAs, or miRNAs. The kit may further comprise packaging and instructions and/or a delivery agent to form a formulation composition. The delivery agent may comprise a saline, a buffered solution, a lipidoid, a dendrimer or any delivery agent disclosed herein. Non-limiting examples of genes include C/EBPα, other members of C/EBP family, albumin gene, alphafectoprotein gene, liver specific factor genes, growth factors, nuclear factor genes, tumor suppressing genes, pluripotency factor genes.

In one non-limiting example, the buffer solution may include sodium chloride, calcium chloride, phosphate and/or EDTA. In another non-limiting example, the buffer solution may include, but is not limited to, saline, saline with 2 mM calcium, 5% sucrose, 5% sucrose with 2 mM calcium, 5% Mannitol, 5% Mannitol with 2 mM calcium, Ringer's lactate, sodium chloride, sodium chloride with 2 mM calcium and mannose (See U.S. Pub. No. 20120258046; herein incorporated by reference in its entirety). In yet another non-limiting example, the buffer solutions may be precipitated, or it may be lyophilized. The amount of each component may be varied to enable consistent, reproducible higher concentration saline or simple buffer formulations. The components may also be varied in order to increase the stability of saRNA in the buffer solution over a period of time and/or under a variety of conditions.

In another embodiment, the present disclosure provides kits to regulate the proliferation of cells, comprising C/EBPα-saRNA of the present disclosure, provided in an amount effective to inhibit the proliferation of cells when introduced into said cells; optionally siRNAs and miRNAs to further regulate the proliferation of target cells; and packaging and instructions and/or a delivery agent to form a formulation composition.

In another embodiment, the present disclosure provides kits for reducing LDL levels in cells, comprising saRNA molecules of the present disclosure; optionally LDL reducing drugs; and packaging and instructions and/or a delivery agent to form a formulation composition.

In another embodiment, the present disclosure provides kits for regulating miRNA expression levels in cells, comprising C/EBPα-saRNA of the present disclosure; optionally siRNAs, eRNAs and lncRNAs; and packaging and instructions and/or a delivery agent to form a formulation composition.

In another embodiment, the present disclosure provides kits for combinational therapies comprising C/EBPα-saRNA of the present disclosure and at least one other active ingredient or therapy.

### Devices

The present disclosure provides for devices which may incorporate C/EBPα-saRNA of the present disclosure. These devices contain in a stable formulation available to be immediately delivered to a subject in need thereof, such as a human patient. Non-limiting examples of such a subject include a subject with hyperproliferative disorders such as cancer, tumor, or liver cirrhosis; and metabolics disorders such as NAFLD, obesity, high LDL cholesterol, or type II diabetes.

In some embodiments, the device contains ingredients in combinational therapies comprising C/EBPα-saRNA of the present disclosure and at least one other active ingredient or therapy.

Non-limiting examples of the devices include a pump, a catheter, a needle, a transdermal patch, a pressurized olfactory delivery device, iontophoresis devices, multi-layered microfluidic devices. The devices may be employed to deliver C/EBPα-saRNA of the present disclosure according to single, multi- or split-dosing regiments. The devices may be employed to deliver C/EBPα-saRNA of the present disclosure across biological tissue, intradermal, subcutaneously, or intramuscularly. More examples of devices suitable for delivering oligonucleotides are disclosed in International Publication WO 2013/090648 filed Dec. 14, 2012, the contents of which are incorporated herein by reference in their entirety.

### Definitions

For convenience, the meaning of certain terms and phrases used in the specification, examples, and appended claims, are provided below. If there is an apparent discrepancy between the usage of a term in other parts of this specification and its definition provided in this section, the definition in this section shall prevail.

About: As used herein, the term “about” means+/−10% of the recited value.

Administered in combination: As used herein, the term “administered in combination” or “combined administration” means that two or more agents, e.g., saRNA, are administered to a subject at the same time or within an interval such that there may be an overlap of an effect of each agent on the patient. In some embodiments, they are administered within about 60, 30, 15, 10, 5, or 1 minute of one another. In some embodiments, the administrations of the agents are spaced sufficiently close together such that a combinatorial (e.g., a synergistic) effect is achieved.

Amino acid: As used herein, the terms “amino acid” and “amino acids” refer to all naturally occurring L-alpha-amino acids. The amino acids are identified by either the one-letter or three-letter designations as follows: aspartic acid (Asp:D), isoleucine threonine (Thr:T), leucine (Leu:L), serine (Ser:S), tyrosine (Tyr:Y), glutamic acid (Glu:E), phenylalanine (Phe:F), proline (Pro:P), histidine (His:H), glycine (Gly:G), lysine (Lys:K), alanine (Ala:A), arginine (Arg:R), cysteine (Cys:C), tryptophan (Trp:W), valine (Val:V), glutamine (Gln:Q) methionine (Met:M), asparagines (Asn:N), where the amino acid is listed first followed parenthetically by the three and one letter codes, respectively.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

Bifunctional: As used herein, the term “bifunctional” refers to any substance, molecule or moiety which is capable of or maintains at least two functions. The functions may affect the same outcome or a different outcome. The structure that produces the function may be the same or different.

Biocompatible: As used herein, the term “biocompatible” means compatible with living cells, tissues, organs or systems posing little to no risk of injury, toxicity or rejection by the immune system.

Biodegradable: As used herein, the term “biodegradable” means capable of being broken down into innocuous products by the action of living things.

Biologically active: As used herein, the phrase “biologically active” refers to a characteristic of any substance that has activity in a biological system and/or organism. For instance, a substance that, when administered to an organism, has a biological effect on that organism, is considered to be biologically active. In particular embodiments, the saRNA of the present disclosure may be considered biologically active if even a portion of the saRNA is biologically active or mimics an activity considered biologically relevant.

Cancer: As used herein, the term “cancer” in an individual refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an individual, or may circulate in the blood stream as independent cells, such as leukemic cells.

Cell growth: As used herein, the term “cell growth” is principally associated with growth in cell numbers, which occurs by means of cell reproduction (i.e. proliferation) when the rate of the latter is greater than the rate of cell death (e.g. by apoptosis or necrosis), to produce an increase in the size of a population of cells, although a small component of that growth may in certain circumstances be due also to an increase in cell size or cytoplasmic volume of individual cells. An agent that inhibits cell growth can thus do so by either inhibiting proliferation or stimulating cell death, or both, such that the equilibrium between these two opposing processes is altered.

Cell type: As used herein, the term “cell type” refers to a cell from a given source (e.g., a tissue, organ) or a cell in a given state of differentiation, or a cell associated with a given pathology or genetic makeup.

Chromosome: As used herein, the term “chromosome” refers to an organized structure of DNA and protein found in cells.

Complementary: As used herein, the term “complementary” as it relates to nucleic acids refers to hybridization or base pairing between nucleotides or nucleic acids, such as, for example, between the two strands of a double-stranded DNA molecule or between an oligonucleotide probe and a target are complementary.

Condition: As used herein, the term “condition” refers to the status of any cell, organ, organ system or organism. Conditions may reflect a disease state or simply the physiologic presentation or situation of an entity. Conditions may be characterized as phenotypic conditions such as the macroscopic presentation of a disease or genotypic conditions such as the underlying gene or protein expression profiles associated with the condition. Conditions may be benign or malignant.

Controlled Release: As used herein, the term “controlled release” refers to a pharmaceutical composition or compound release profile that conforms to a particular pattern of release to effect a therapeutic outcome.

Cytostatic: As used herein, “cytostatic” refers to inhibiting, reducing, suppressing the growth, division, or multiplication of a cell (e.g., a mammalian cell (e.g., a human cell)), bacterium, virus, fungus, protozoan, parasite, prion, or a combination thereof.

Cytotoxic: As used herein, “cytotoxic” refers to killing or causing injurious, toxic, or deadly effect on a cell (e.g., a mammalian cell (e.g., a human cell)), bacterium, virus, fungus, protozoan, parasite, prion, or a combination thereof.

Delivery: As used herein, “delivery” refers to the act or manner of delivering a compound, substance, entity, moiety, cargo or payload.

Delivery Agent: As used herein, “delivery agent” refers to any substance which facilitates, at least in part, the in vivo delivery of a saRNA of the present disclosure to targeted cells.

Destabilized: As used herein, the term “destable,” “destabilize,” or “destabilizing region” means a region or molecule that is less stable than a starting, wild-type or native form of the same region or molecule.

Detectable label: As used herein, “detectable label” refers to one or more markers, signals, or moieties which are attached, incorporated or associated with another entity that is readily detected by methods known in the art including radiography, fluorescence, chemiluminescence, enzymatic activity, absorbance and the like. Detectable labels include radioisotopes, fluorophores, chromophores, enzymes, dyes, metal ions, ligands such as biotin, avidin, streptavidin and haptens, quantum dots, and the like. Detectable labels may be located at any position in the peptides, proteins or polynucleotides, e.g, saRNA, disclosed herein. They may be within the amino acids, the peptides, proteins, or polynucleotides located at the N- or C-termini or 5′ or 3′ termini as the case may be.

Encapsulate: As used herein, the term “encapsulate” means to enclose, surround or encase.

Engineered: As used herein, embodiments of the disclosure are “engineered” when they are designed to have a feature or property, whether structural or chemical, that varies from a starting point, wild type or native molecule.

Equivalent subject: As used herein, “equivalent subject” may be e.g. a subject of similar age, sex and health such as liver health or cancer stage, or the same subject prior to treatment according to the disclosure. The equivalent subject is “untreated” in that he does not receive treatment with a saRNA according to the disclosure. However, he may receive a conventional anti-cancer treatment, provided that the subject who is treated with the saRNA of the disclosure receives the same or equivalent conventional anti-cancer treatment.

Exosome: As used herein, “exosome” is a vesicle secreted by mammalian cells.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Feature: As used herein, a “feature” refers to a characteristic, a property, or a distinctive element.

Formulation: As used herein, a “formulation” includes at least a saRNA of the present disclosure and a delivery agent.

Fragment: A “fragment,” as used herein, refers to a portion. For example, fragments of proteins may comprise polypeptides obtained by digesting full-length protein isolated from cultured cells.

Functional: As used herein, a “functional” biological molecule is a biological molecule in a form in which it exhibits a property and/or activity by which it is characterized.

Gene: As used herein, the term “gene” refers to a nucleic acid sequence that comprises control and most often coding sequences necessary for producing a polypeptide or precursor. Genes, however, may not be translated and instead code for regulatory or structural RNA molecules.

A gene may be derived in whole or in part from any source known to the art, including a plant, a fungus, an animal, a bacterial genome or episome, eukaryotic, nuclear or plasmid DNA, cDNA, viral DNA, or chemically synthesized DNA. A gene may contain one or more modifications in either the coding or the untranslated regions that could affect the biological activity or the chemical structure of the expression product, the rate of expression, or the manner of expression control. Such modifications include, but are not limited to, mutations, insertions, deletions, and substitutions of one or more nucleotides. The gene may constitute an uninterrupted coding sequence or it may include one or more introns, bound by the appropriate splice junctions.

Gene expression: As used herein, the term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

Genome: The term “genome” is intended to include the entire DNA complement of an organism, including the nuclear DNA component, chromosomal or extrachromosomal DNA, as well as the cytoplasmic domain (e.g., mitochondrial DNA).

Homology: As used herein, the term “homology” refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the disclosure, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the disclosure, two protein sequences are considered to be homologous if the proteins are at least about 50%, 60%, 70%, 80%, or 90% identical for at least one stretch of at least about 20 amino acids.

The term “hyperproliferative cell” may refer to any cell that is proliferating at a rate that is abnormally high in comparison to the proliferating rate of an equivalent healthy cell (which may be referred to as a “control”). An “equivalent healthy” cell is the normal, healthy counterpart of a cell. Thus, it is a cell of the same type, e.g. from the same organ, which performs the same functions(s) as the comparator cell. For example, proliferation of a hyperproliferative hepatocyte should be assessed by reference to a healthy hepatocyte, whereas proliferation of a hyperproliferative prostate cell should be assessed by reference to a healthy prostate cell.

By an “abnormally high” rate of proliferation, it is meant that the rate of proliferation of the hyperproliferative cells is increased by at least 20, 30, 40%, or at least 45, 50, 55, 60, 65, 70, 75%, or at least 80%, as compared to the proliferative rate of equivalent, healthy (non-hyperproliferative) cells. The “abnormally high” rate of proliferation may also refer to a rate that is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, compared to the proliferative rate of equivalent, healthy cells.

The term “hyperproliferative cell” as used herein does not refer to a cell which naturally proliferates at a higher rate as compared to most cells, but is a healthy cell. Examples of cells that are known to divide constantly throughout life are skin cells, cells of the gastrointestinal tract, blood cells and bone marrow cells. However, when such cells proliferate at a higher rate than their healthy counterparts, then they are hyperproliferative.

Hyperproliferative disorder: As used herein, a “hyperproliferative disorder” may be any disorder which involves hyperproliferative cells as defined above. Examples of hyperproliferative disorders include neoplastic disorders such as cancer, psoriatic arthritis, rheumatoid arthritis, gastric hyperproliferative disorders such as inflammatory bowel disease, skin disorders including psoriasis, Reiter's syndrome, pityriasis rubra pilaris, and hyperproliferative variants of the disorders of keratinization.

The skilled person is fully aware of how to identify a hyperproliferative cell. The presence of hyperproliferative cells within an animal may be identifiable using scans such as X-rays, MRI or CT scans. The hyperproliferative cell may also be identified, or the proliferation of cells may be assayed, through the culturing of a sample in vitro using cell proliferation assays, such as MTT, XTT, MTS or WST-1 assays. Cell proliferation in vitro can also be determined using flow cytometry.

Identity: As used herein, the term “identity” refers to the overall relatedness between polymeric molecules, e.g., between oligonucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, N.J., 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically a reduction in the level of an mRNA results in a reduction in the level of a polypeptide translated therefrom. The level of expression may be determined using standard techniques for measuring mRNA or protein.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. Substantially isolated: By “substantially isolated” is meant that the compound is substantially separated from the environment in which it was formed or detected. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art.

Label: The term “label” refers to a substance or a compound which is incorporated into an object so that the substance, compound or object may be detectable.

Linker: As used herein, a linker refers to a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. The linker can be attached to a modified nucleoside or nucleotide on the nucleobase or sugar moiety at a first end, and to a payload, e.g., a detectable or therapeutic agent, at a second end. The linker may be of sufficient length as to not interfere with incorporation into a nucleic acid sequence. The linker can be used for any useful purpose, such as to form saRNA conjugates, as well as to administer a payload, as described herein. Examples of chemical groups that can be incorporated into the linker include, but are not limited to, alkyl, alkenyl, alkynyl, amido, amino, ether, thioether, ester, alkylene, heteroalkylene, aryl, or heterocyclyl, each of which can be optionally substituted, as described herein. Examples of linkers include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers and derivatives thereof. Other examples include, but are not limited to, cleavable moieties within the linker, such as, for example, a disulfide bond (—S—S—) or an azo bond (—N═N—), which can be cleaved using a reducing agent or photolysis. Non-limiting examples of a selectively cleavable bond include an amido bond can be cleaved for example by the use of tris(2-carboxyethyl)phosphine (TCEP), or other reducing agents, and/or photolysis, as well as an ester bond can be cleaved for example by acidic or basic hydrolysis.

Metastasis: As used herein, the term “metastasis” means the process by which cancer spreads from the place at which it first arose as a primary tumor to distant locations in the body. Metastasis also refers to cancers resulting from the spread of the primary tumor. For example, someone with breast cancer may show metastases in their lymph system, liver, bones or lungs.

Modified: As used herein “modified” refers to a changed state or structure of a molecule of the disclosure. Molecules may be modified in many ways including chemically, structurally, and functionally. In one embodiment, the saRNA molecules of the present disclosure are modified by the introduction of non-natural nucleosides and/or nucleotides.

Naturally occurring: As used herein, “naturally occurring” means existing in nature without artificial aid.

Nucleic acid: The term “nucleic acid” as used herein, refers to a molecule comprised of one or more nucleotides, i.e., ribonucleotides, deoxyribonucleotides, or both. The term includes monomers and polymers of ribonucleotides and deoxyribonucleotides, with the ribonucleotides and/or deoxyribonucleotides being bound together, in the case of the polymers, via 5′ to 3′ linkages. The ribonucleotide and deoxyribonucleotide polymers may be single or double-stranded. However, linkages may include any of the linkages known in the art including, for example, nucleic acids comprising 5′ to 3′ linkages. The nucleotides may be naturally occurring or may be synthetically produced analogs that are capable of forming base-pair relationships with naturally occurring base pairs. Examples of non-naturally occurring bases that are capable of forming base-pairing relationships include, but are not limited to, aza and deaza pyrimidine analogs, aza and deaza purine analogs, and other heterocyclic base analogs, wherein one or more of the carbon and nitrogen atoms of the pyrimidine rings have been substituted by heteroatoms, e.g., oxygen, sulfur, selenium, phosphorus, and the like.

Patient: As used herein, “patient” refers to a subject who may seek or be in need of treatment, requires treatment, is receiving treatment, will receive treatment, or a subject who is under care by a trained professional for a particular disease or condition.

Peptide: As used herein, “peptide” is less than or equal to 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Pharmaceutically acceptable solvate: The term “pharmaceutically acceptable solvate,” as used herein, means a compound of the disclosure wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

Pharmacologic effect: As used herein, a “pharmacologic effect” is a measurable biologic phenomenon in an organism or system which occurs after the organism or system has been contacted with or exposed to an exogenous agent. Pharmacologic effects may result in therapeutically effective outcomes such as the treatment, improvement of one or more symptoms, diagnosis, prevention, and delay of onset of disease, disorder, condition or infection. Measurement of such biologic phenomena may be quantitative, qualitative or relative to another biologic phenomenon. Quantitative measurements may be statistically significant. Qualitative measurements may be by degree or kind and may be at least 10%, 20%, 30%, 40%, 50%, 60%, 70%, 80%, 90% or more different. They may be observable as present or absent, better or worse, greater or less. Exogenous agents, when referring to pharmacologic effects are those agents which are, in whole or in part, foreign to the organism or system. For example, modifications to a wild type biomolecule, whether structural or chemical, would produce an exogenous agent. Likewise, incorporation or combination of a wild type molecule into or with a compound, molecule or substance not found naturally in the organism or system would also produce an exogenous agent. The saRNA of the present disclosure, comprises exogenous agents. Examples of pharmacologic effects include, but are not limited to, alteration in cell count such as an increase or decrease in neutrophils, reticulocytes, granulocytes, erythrocytes (red blood cells), megakaryocytes, platelets, monocytes, connective tissue macrophages, epidermal langerhans cells, osteoclasts, dendritic cells, microglial cells, neutrophils, eosinophils, basophils, mast cells, helper T cells, suppressor T cells, cytotoxic T cells, natural killer T cells, B cells, natural killer cells, or reticulocytes. Pharmacologic effects also include alterations in blood chemistry, pH, hemoglobin, hematocrit, changes in levels of enzymes such as, but not limited to, liver enzymes AST and ALT, changes in lipid profiles, electrolytes, metabolic markers, hormones or other marker or profile known to those of skill in the art.

Physicochemical: As used herein, “physicochemical” means of or relating to a physical and/or chemical property.

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Prodrug: The present disclosure also includes prodrugs of the compounds described herein. As used herein, “prodrugs” refer to any substance, molecule or entity which is in a form predicate for that substance, molecule or entity to act as a therapeutic upon chemical or physical alteration. Prodrugs may by covalently bonded or sequestered in some way and which release or are converted into the active drug moiety prior to, upon or after administered to a mammalian subject. Prodrugs can be prepared by modifying functional groups present in the compounds in such a way that the modifications are cleaved, either in routine manipulation or in vivo, to the parent compounds. Prodrugs include compounds wherein hydroxyl, amino, sulfhydryl, or carboxyl groups are bonded to any group that, when administered to a mammalian subject, cleaves to form a free hydroxyl, amino, sulfhydryl, or carboxyl group respectively. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference in their entirety.

Prognosing: As used herein, the term “prognosing” means a statement or claim that a particular biologic event will, or is very likely to, occur in the future.

Progression: As used herein, the term “progression” or “cancer progression” means the advancement or worsening of or toward a disease or condition.

Proliferate: As used herein, the term “proliferate” means to grow, expand or increase or cause to grow, expand or increase rapidly. “Proliferative” means having the ability to proliferate. “Anti-proliferative” means having properties counter to or inapposite to proliferative properties.

Protein: A “protein” means a polymer of amino acid residues linked together by peptide bonds. The term, as used herein, refers to proteins, polypeptides, and peptides of any size, structure, or function. Typically, however, a protein will be at least 50 amino acids long. In some instances the protein encoded is smaller than about 50 amino acids. In this case, the polypeptide is termed a peptide. If the protein is a short peptide, it will be at least about 10 amino acid residues long. A protein may be naturally occurring, recombinant, or synthetic, or any combination of these. A protein may also comprise a fragment of a naturally occurring protein or peptide. A protein may be a single molecule or may be a multi-molecular complex. The term protein may also apply to amino acid polymers in which one or more amino acid residues are an artificial chemical analogue of a corresponding naturally occurring amino acid.

Protein expression: The term “protein expression” refers to the process by which a nucleic acid sequence undergoes translation such that detectable levels of the amino acid sequence or protein are expressed.

Purified: As used herein, “purify,” “purified,” “purification” means to make substantially pure or clear from unwanted components, material defilement, admixture or imperfection.

Regression: As used herein, the term “regression” or “degree of regression” refers to the reversal, either phenotypically or genotypically, of a cancer progression. Slowing or stopping cancer progression may be considered regression.

Sample: As used herein, the term “sample” or “biological sample” refers to a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen). A sample further may include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule.

Signal Sequences: As used herein, the phrase “signal sequences” refers to a sequence which can direct the transport or localization of a protein.

Single unit dose: As used herein, a “single unit dose” is a dose of any therapeutic administered in one dose/at one time/single route/single point of contact, i.e., single administration event.

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Split dose: As used herein, a “split dose” is the division of single unit dose or total daily dose into two or more doses.

Stable: As used herein “stable” refers to a compound that is sufficiently robust to survive isolation to a useful degree of purity from a reaction mixture, and preferably capable of formulation into an efficacious therapeutic agent.

Stabilized: As used herein, the term “stabilize”, “stabilized,” “stabilized region” means to make or become stable.

Subject: As used herein, the term “subject” or “patient” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneously: As used herein and as it relates to plurality of doses, the term means within 2 seconds.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Sustained release: As used herein, the term “sustained release” refers to a pharmaceutical composition or compound release profile that conforms to a release rate over a specific period of time.

Synthetic: The term “synthetic” means produced, prepared, and/or manufactured by the hand of man. Synthesis of polynucleotides or polypeptides or other molecules of the present disclosure may be chemical or enzymatic.

Targeted Cells: As used herein, “targeted cells” refers to any one or more cells of interest. The cells may be found in vitro, in vivo, in situ or in the tissue or organ of an organism. The organism may be an animal, preferably a mammal, more preferably a human and most preferably a patient.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Total daily dose: As used herein, a “total daily dose” is an amount given or prescribed in 24 hr period. It may be administered as a single unit dose.

Transcription factor: As used herein, the term “transcription factor” refers to a DNA-binding protein that regulates transcription of DNA into RNA, for example, by activation or repression of transcription. Some transcription factors effect regulation of transcription alone, while others act in concert with other proteins. Some transcription factor can both activate and repress transcription under certain conditions. In general, transcription factors bind a specific target sequence or sequences highly similar to a specific consensus sequence in a regulatory region of a target gene. Transcription factors may regulate transcription of a target gene alone or in a complex with other molecules.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

The phrase “a method of treating” or its equivalent, when applied to, for example, cancer refers to a procedure or course of action that is designed to reduce, eliminate or prevent the number of cancer cells in an individual, or to alleviate the symptoms of a cancer. “A method of treating” cancer or another proliferative disorder does not necessarily mean that the cancer cells or other disorder will, in fact, be completely eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of an individual, is nevertheless deemed an overall beneficial course of action.

Tumor growth: As used herein, the term “tumor growth” or “tumor metastases growth”, unless otherwise indicated, is used as commonly used in oncology, where the term is principally associated with an increased mass or volume of the tumor or tumor metastases, primarily as a result of tumor cell growth.

Tumor Burden: As used herein, the term “tumor burden” refers to the total Tumor Volume of all tumor nodules with a diameter in excess of 3 mm carried by a subject.

Tumor Volume: As used herein, the term “tumor volume” refers to the size of a tumor. The tumor volume in mm³ is calculated by the formula: volume=(width)²×length/2.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

### Equivalents and Scope

Those skilled in the art will recognize, or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

It is also noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any nucleic acid or protein encoded thereby; any method of production; any method of use; etc.) can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

The disclosure is further illustrated by the following non-limiting examples.

### EXAMPLES

### Example 1. Preparations of CEBPA-51 and MTL-CEBPA

Materials and Procedures of preparing CEBPA-saRNAs have been disclosed in WO2015/075557 and WO2016/170349 to MiNA Therapeutics Limited. The preparations of CEBPA-51 and MTL-CEBPA have been disclosed in Examples of WO2016/170349.

In brief, each strand of CEBPA-51 was synthesized on a solid support by coupling phosphoramidite monomers sequentially. The synthesis was performed on an automatic synthesizer such as an Akta Oligopilot 100 (GE Healthcare) and a Technikrom synthesizer (Asahi Kasei Bio) that delivers specified volumes of reagents and solvents to and from the synthesis reactor (column type) packed with solid support. The process began with charging reagents to the designated reservoirs connected to the reactor and packing of the reactor vessel with the appropriate solid support. The flow of reagent and solvents was regulated by a series of computer-controlled valves and pumps with automatic recording of flow rate and pressure. The solid-phase approach enabled efficient separation of reaction products as coupled to the solid phase from reagents in solution phase at each step in the synthesis by washing of the solid support with solvent.

CEBPA-51 was dissolved at ambient temperature in sodium acetate/sucrose buffer pH 4.0 and lipids were dissolved in absolute ethanol at 55° C. Liposomes were prepared by crossflow ethanol injection technology. Immediately after liposome formation, the suspension was diluted with sodium chloride/phosphate buffer pH 9.0. The collected intermediate product was extruded through polycarbonate membranes with a pore size of 0.2 μm. The target saRNA concentration was achieved by ultrafiltration. Non-encapsulated drug substance and residual ethanol were removed by subsequent diafiltration with sucrose/phosphate buffer pH 7.5. Thereafter, the concentrated liposome suspension was 0.2 μm filtrated and stored at 5±3° C. Finally, the bulk product was formulated, 0.2 μm filtrated and filled in 20 ml vials.

MTL-CEBPA was presented as a concentrate solution for infusion. Each vial contains 50 mg of CEBPA-51 (saRNA) in 20 ml of sucrose/phosphate buffer pH about 7.5.

### Example 2. CEBPA-saRNA Inhibits Immune Suppression of MDSC

### Materials and Methods

### Cell Lines

LLC lung carcinoma cell line was obtained from ATCC and cultured in DMEM (Corning Incorporated) supplemented with 10% FBS (Atlanta Biologicals, Inc.) and 1% antibiotics (Thermo Fisher Scientific Inc.). Cells were incubated in a 37° C. and 5% CO₂. 70-80% confluent cells were harvested using 0.25% Trypsin (Thermo Fisher Scientific Inc.) and passaged or used for experiments.

### Animals

All procedures were performed and approved in strict accordance with the Institutional Animal Care and Use Committee (IACUC) at The Wistar Institute, and with the NIH Guide for the Care and Use of Laboratory Animal guidelines. Female six-week old C57BL/6 mice (Charles River Labs) were maintained in a temperature-controlled room with a 12/12 hour light/dark schedule and food provided ad libitum.

### Tumor Bearing Mice and Treatment

The LLC cells were harvested and suspended in DPBS (Corning) as 200 μL containing 5×10⁵ cells, and injected s.c. into the mice on Day 0. After tumors were established, the mice were randomized into 2 groups and intravenously treated with 3 mg/kg of MTL-CEBPA or NOV-FLUC twice a week.

### Cell Isolation

The tumor-bearing mice treated with MTL-CEBPA or NOV-FLUC were sacrificed on Day 24 and 25. The tumor tissues were dissociated using tumor dissociation kit (Miltenyi Biotec). Spleens were processed by physically mashing. Red blood cells were lysed by ACK buffer.

### Flow Cytometry

Monoclonal antibodies specific to the mouse cell surface markers CD45, CD11b, Ly6G, Ly6C, F4/80 were purchased from BD bioscience. Flow cytometry data were acquired using a BD LSR II flow cytometer and analyzed using FlowJo software (Tree Star).

### Suppression Assay

PMN-MDSC (CD11b+, Ly6G+, Ly6Clow), M-MDSC (CD11b+, Ly6G−, Ly6Chigh) and macrophage (CD11b+, F4/80+) were isolated from tumor cells by cell sorting on FACSAria cell sorter (BD Biosciences). PMEL mice have CD8+ T cells which recognize gp100-derived peptide, were used as responders. Whole spleen cells from PMEL mice were mixed with spleen cells from naïve mice at 1:4 in complete RPMI media and plated into 96-well U-bottom plates at 10⁵ cells/well. Ly6G+ or Ly6C+ cells were added to the wells at 0.0625-1×10⁵ cells/well (1:16-1:1). Murine gp100 peptide (25-33) EGSRNQDWL (AnaSpec, Inc.) was solved in ddH₂O, diluted with RPMI complete media and added into the wells at the final concentration of 0.1 μg/mL. After 48 hours of cultivation, cells were pulsed with ³H-thymidine (1 μCi/well; GE healthcare) for 16 hours. ³H-thymidine uptake was counted using a liquid scintillation counter as counts per minute (cpm) and calculated the percentage of proliferation to the positive control (the wells with responder cells and peptide).

### Quantitative RT-PCR

RNA was extracted with total RNA extraction kit (Zymo research). cDNA was synthesized (cDNA reverse transcriptase kit; Applied Biosystems), and PCR was performed in triplicate for each sample using SYBR Green Master Mixture (Thermo Fisher) and primers for b-actin (Fwd: 5′-ATGGAGGGGAATACAGCCC-3′, Rev: 5′-TTCTTTGCAGCTCCTTCGTT-3′), lactotransferrin (Ltf, Fwd: 5′-TGCTCCCAACAGCAAAGAGA-3′, Rev: 5′-CTTCAGTGTTCTTCCCGTCAGT-3′) and C/EBP-alpha (QuantiTect Primer Assays, Qiagen) Relative expression of the genes compared to b-actin was calculated using the 2^{-ΔCt} method.

### CD8 Depletion

Anti-mouse CD8a antibody or rat IgG2a isotype control (BioXCell) was i.p. administered to the mice at 100 μg/mouse on Day −3, 1, 4, 7, 10, and 14. On Day 0, LLC cells were injected s.c. into the mice at 5×10⁵ cells/mouse. On Day 3, the mice were randomized into 3 groups (n=5) and intravenously treated with 3 mg/kg of MTL-CEBPA or NOV-FLUC twice a week.

### Results and Discussion

The LLC cells were injected s.c. into the mice on Day 0. On Day 3, the mice were randomized into 2 groups and intravenously treated with 3 mg/kg of MTL-CEBPA or NOV-FLUC twice a week. Tumor areas of the mice were measured and shown in FIG. 1. MTL-CEBPA showed tumor growth inhibition.

The tumor-bearing mice treated with MTL-CEBPA or NOV-FLUC were sacrificed on Day 24 and 25. Spleen cells and tumor cells were analyzed by flow cytometry. Myeloid cell proportion of spleen and of tumor were measured.

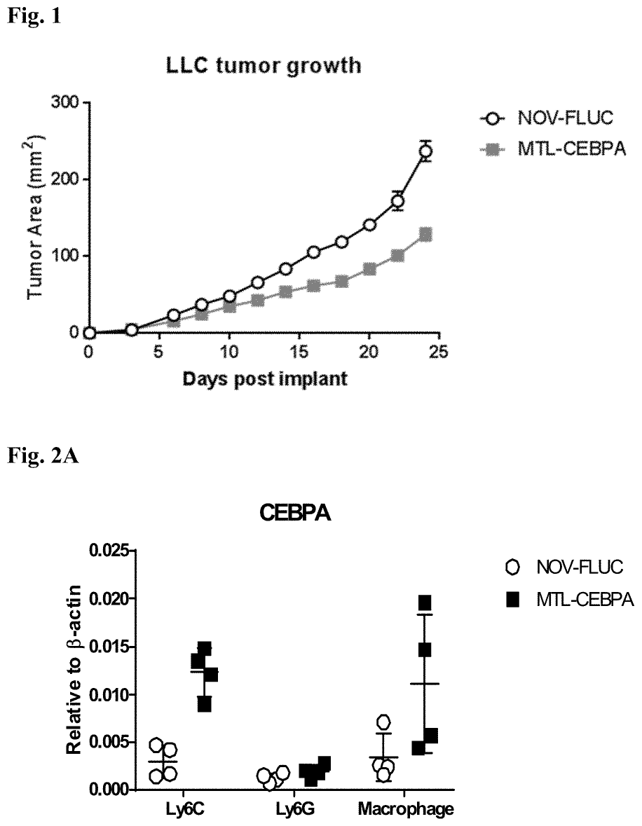

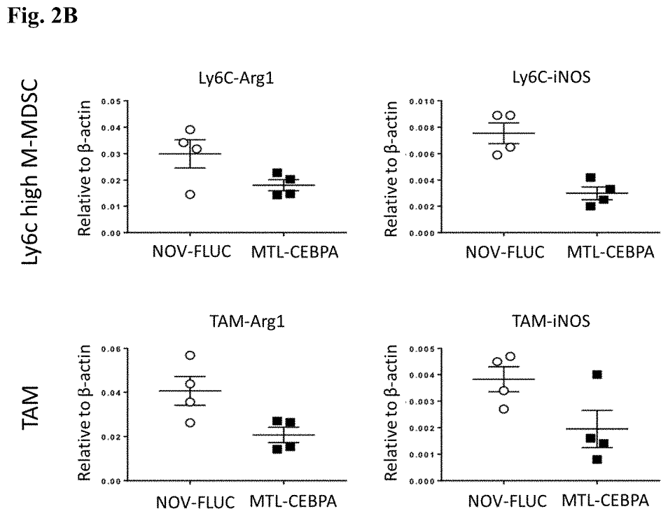

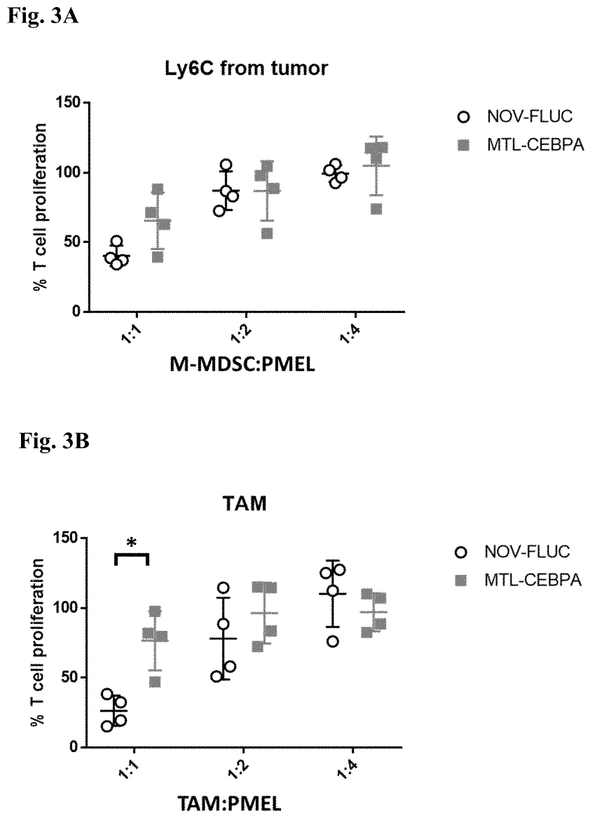

M-MDSC, PMN-MDSC and TAM (tumor associated macrophage) cells were isolated from tumor cells by cell sorting on FACSAria (BD Biosciences). Total RNA was extracted and the expressions of genes were analyzed by qRT-PCR. As shown in FIG. 2A, C/EBPα expression was upregulated in M-MDSC, PMN-MDSC and TAM cells. Ly6C stands for M-MDSC cells, Ly6G for PMN-MDSC cells. As shown in FIG. 2B, ARG1 and iNOS gene expressions reduced. Using PMEL mice as a responder cells, suppression assay was performed. Serial dilutions of M-MDSC and TAM were performed and % T cell proliferation was measured and shown in FIG. 3A and FIG. 3B. Suppressive activities of M-MDSC and TAM from tumor were observed. MTL-CEBPA treated and untreated cells showed different activities.

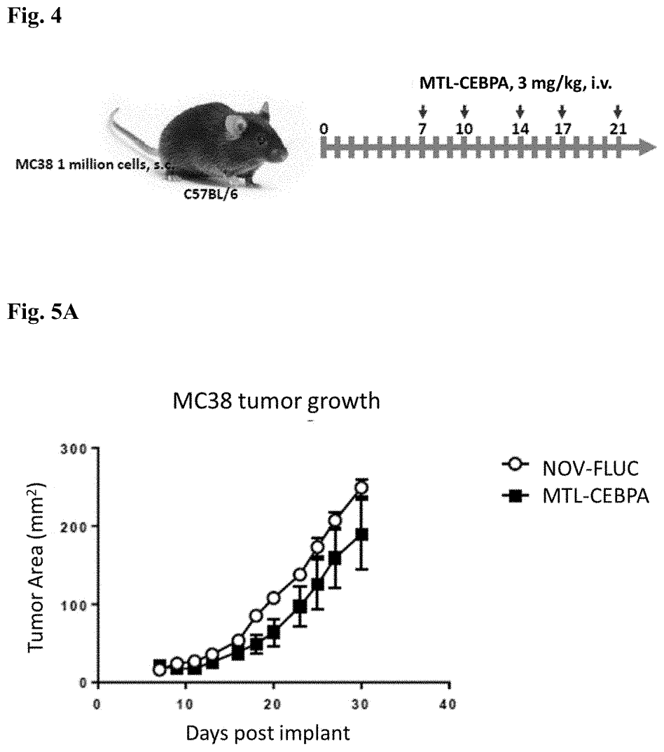

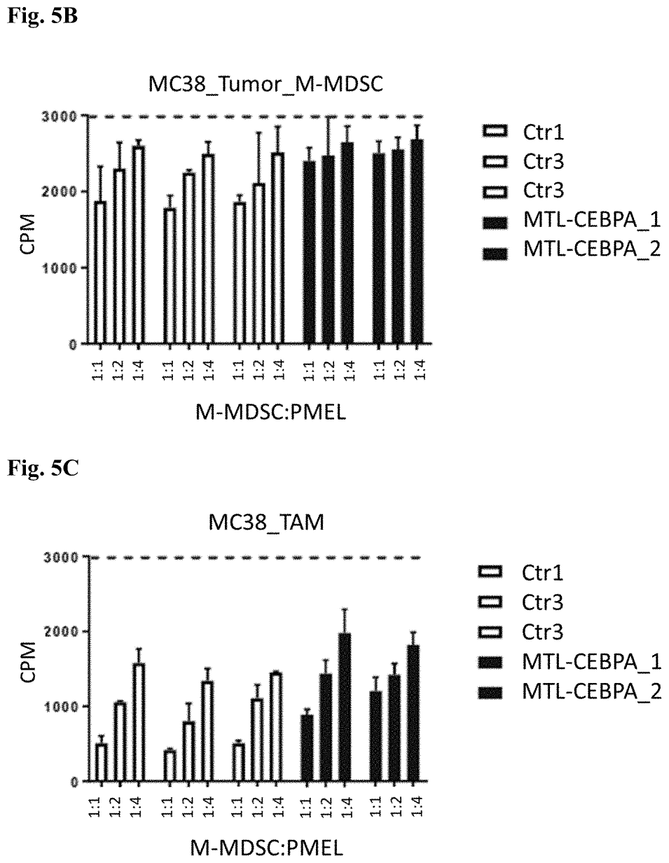

A similar study was carried out in MC38 model (immune responsive colon cancer). The study design was summarized in FIG. 4. Tumor areas after MTL-CEBPA treatment were measured and shown in FIG. 5A. MTL-CEBPA showed efficacy to reduce tumor areas compared to control. T cell proliferations (count per min (CPM)) in M-MDSC and TAM cells are shown in FIG. 5B and FIG. 5C. M-MDSC and TAM from the tumors of MTL-CEBPA-treated mice had less suppressive activity against T cell proliferation.

Therefore, MTL-CEBPA can be used to upregulate C/EBPα expression and downregulate ARG1 and iNOS expressions in M-MDSC, PMN-MDSC and TAM cells. MTL-CEBPA can also be used to block M-MDSC's and TAM's inhibitory activity of T-cell proliferation.

### Example 3. CEBPA-saRNA Combination Therapies with Anti-CTLA4 Ab and COX2 Inhibitor

As discussed above, CEBPA-saRNAs can be used to reduce the immune suppression of MDSC and TAM cells. In this study, MTL-CEBPA was combined with various immune therapies. The study design was summarized in FIG. 6. The tumor-bearing mice (LLC model) were separated into groups and treated with: Group 1): control, Group 2): MTL-CEBPA at 3 mg/kg (i.v.) on days 3, 6, 10, 13, 17, and 20, Group 3): CTLA4 antibody (Ab) that inhibitors CTLA4 activity at 200 ug/mouse (i.p.) on days 10, 17 and 24, Group 4): Celecoxib, a COX2 inhibitor, at 50 mg/kg (p.o.) everyday, Group 5): MTL-CEBPA+CTLA4 Ab, and Group 6): MTL-CEBPA+Celecoxib.

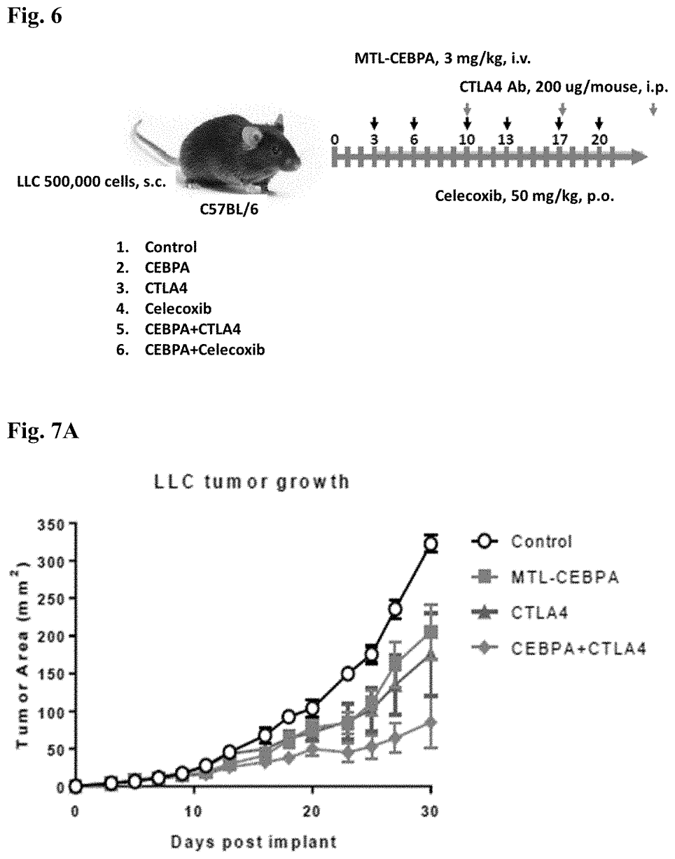

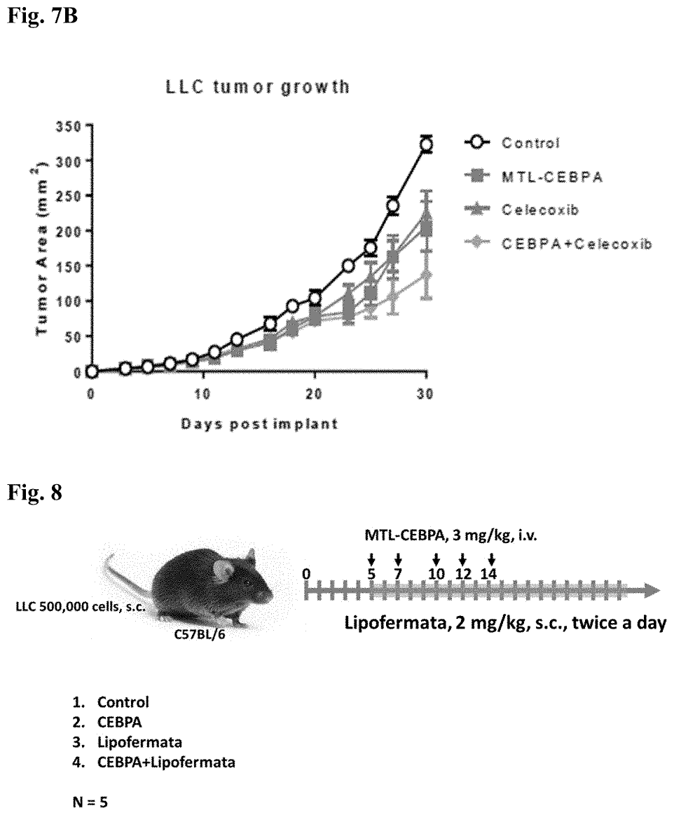

Tumors areas after treatments were measured. As shown in FIG. 7A, the combination therapy of MTL-CEBPA+CTLA4 Ab had the best tumor inhibition compared with single agent treatments. As shown in FIG. 7B, the combination therapy of MTL-CEBPA+Celecoxib also had the best tumor inhibition compared with single agent treatments.

### Example 4. CEBPA-saRNA Combination Therapies with Lipofermata

Polymorphonuclear myeloid-derived suppressor cells (PMN-MDSCs) are pathologically activated neutrophils that are crucial for the regulation of immune responses in cancer. These cells contribute to the failure of cancer therapies and are associated with poor clinical outcomes. It has been reported that mouse and human PMN-MDSCs upregulate fatty acid transport protein 2 (FATP2) and the inhibition of FATP2 abrogated the activity of PMN-MDSCs and substantially delayed tumour progression.

In this study, MTL-CEBPA was combined with Lipofermata ((5-bromo-5′-phenylspiro[3H-1,3,4-thiadiazole-2,3′-indoline]-2-one)), a FATP2 inhibitor. The study design was summarized in FIG. 8. The tumor-bearing mice (LLC model) were separated into groups and treated with: Group 1): control, Group 2): MTL-CEBPA at 3 mg/kg (i.v.) on days 5, 7, 10, 12 and 14, Group 3): Lipofermata at 2 mg/kg (s.c.) twice a day on each day, Group 4): MTL-CEBPA 3 mg/kg (i.v.) on days 5, 7, 10, 12 and 14+Lipofermata 2 mg/kg (s.c.) twice a day on each day.

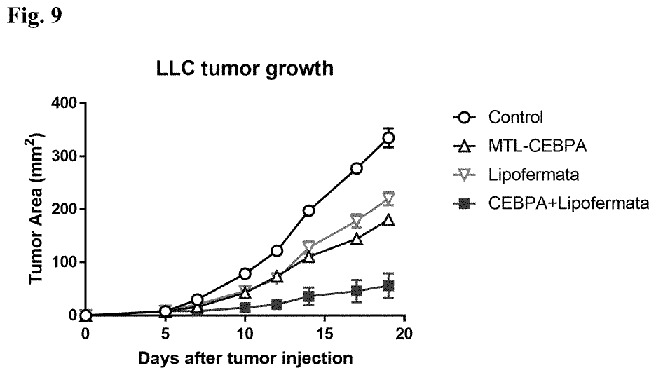

Tumors areas after treatments were measured. As shown in FIG. 9, the combination therapy of MTL-CEBPA+Lipofermata had the best tumor inhibition compared with single agent treatments.

### Equivalents and Scope

Those skilled in the art will recognize or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

1. A method of blocking MDSC's or TAM's inhibitory activity against T-cell proliferation in a subject in need thereof, comprising administering a synthetic isolated saRNA to the subject, wherein the saRNA comprises an antisense strand with a sequence of SEQ ID No. 1 (CEBPA-51).

2. The method of claim 1, wherein the saRNA is double-stranded and further comprises a sense strand.

3. The method of claim 2, wherein the sense strand of the saRNA comprises a sequence of SEQ ID No. 2 (CEBPA-51).

4. The method of claim 3, wherein CEBPA-51 is delivered with liposomes.

5. The method of claim 4, wherein the liposomes are NOV340 Smarticles.

6. The method of claim 1, wherein the T-cell proliferation is up-regulated by at least 20%, 50%, 100%, 2 folds, 3 folds, 4 folds or 5 folds.

7. The method of claim 1, where in the subject has tumor.

8. The method of claim 1, wherein the subject has lung cancer or colon cancer.

9. A method of up-regulating the expression of the C/EBPα gene in a cell in a subject in need thereof, wherein the cell is a monocytic myeloid-derived suppressor cell (MDSC) or a tumor associated macrophage (TAM), comprising administering a synthetic isolated saRNA to the cell, wherein the saRNA comprises an antisense strand with a sequence of SEQ ID No. 1 (CEBPA-51).

10. The method of claim 9, wherein the saRNA is double-stranded and further comprises a sense strand.

11. The method of claim 10, wherein the sense strand of the saRNA comprises a sequence of SEQ ID No. 2 (CEBPA-51).

12. The method of claim 11, wherein CEBPA-51 is delivered with liposomes.

13. The method of claim 12, wherein the liposomes are NOV340 Smarticles.

14. The method of claim 9, wherein the expression of the C/EBPα gene is up-regulated by at least 20%, 50%, 100%, 2 folds, 3 folds, 4 folds or 5 folds.

15. The method of claim 9, where in the subject has tumor.

16. The method of claim 9, wherein the subject has lung cancer or colon cancer.

17. A method of reducing the expression of a target gene in a cell in a subject in need thereof, comprising administering a synthetic isolated saRNA to the cell, wherein the saRNA comprises an antisense strand with a sequence of SEQ ID No. 1 (CEBPA-51), wherein the target gene is ARG1, iNOS, S100A8 or S100A9.

18. The method of claim 17, wherein the saRNA is double-stranded and further comprises a sense strand.

19. The method of claim 18, wherein the sense strand of the saRNA comprises a sequence of SEQ ID No. 2 (CEBPA-51).

20. The method of claim 19, wherein CEBPA-51 is delivered with liposomes.

21. The method of claim 20, wherein the liposomes are NOV340 Smarticles.

22. The method of claim 17, wherein the target gene expression is reduced by at least 10%, 20%, 30%, 40%, 50%, 60%, 70%, or 80%.

23. The method of claim 17, wherein the cell is MDSC or TAM.

24. The method of claim 17, where in the subject has tumor.

25. The method of claim 18, wherein the subject has lung cancer or colon cancer.

26. A method of delivering a synthetic isolated saRNA to myeloid cells of a subject in need thereof, wherein the saRNA comprises an antisense strand with a sequence of SEQ ID No. 1 (CEBPA-51), comprising formulating the saRNA with liposomes.

27. The method of claim 26, wherein the saRNA is double-stranded and further comprises a sense strand.

28. The method of claim 27, wherein the sense strand of the saRNA comprises a sequence of SEQ ID No. 2 (CEBPA-51).

29. The method of claim 26, wherein the liposomes are NOV340 Smarticles.

30. The method of claim 26, where in the subject has tumor.

31. The method of claim 26, wherein the subject has lung cancer or colon cancer.

32. A method of treating cancer in a subjection in need thereof, comprising administering a synthetic isolated saRNA to the cell and an additional active agent, wherein the saRNA comprises an antisense strand with a sequence of SEQ ID No. 1 (CEBPA-51), and wherein the additional active agent is a CTLA-4 inhibitor, a COX2 inhibitor, or a FATP2 inhibitor.

33. The method of claim 32, wherein the saRNA is double-stranded and further comprises a sense strand.

34. The method of claim 33, wherein the sense strand of the saRNA comprises a sequence of SEQ ID No. 2 (CEBPA-51).

35. The method of claim 32, wherein CEBPA-51 is delivered with liposomes.

36. The method of claim 35, wherein the liposomes are NOV340 Smarticles.

37. The method of claim 32, wherein the CTLA-4 inhibitor is a CTLA-4 antibody.

38. The method of claim 32, wherein the COX2 inhibitor is celecoxib.

39. The method of claim 32, wherein the FATP2 inhibitor is lipofermata.

40. The method of claim 32, where the subject has lung cancer or colon cancer.


SEQUENCE LISTING (3 sequences)

SEQ ID NO: 1 - RNA, 21 nt, Artificial Sequence
  Description of Artificial Sequence Synthetic oligonucleotide
  modified_base (20)..(21): 2′-OMe modified nucleotide
gaccagugacaaugaccgcuu

SEQ ID NO: 2 - RNA, 21 nt, Artificial Sequence
  Description of Artificial Sequence Synthetic oligonucleotide
  modified_base (1)..(1): Inverted abasic modification
  modified_base (1)..(2): 2′-OMe modified nucleotide
  modified_base (4)..(4): 2′-OMe modified nucleotide
  modified_base (10)..(10): 2′-OMe modified nucleotide
  modified_base (14)..(14): 2′-OMe modified nucleotide
  modified_base (20)..(21): 2′-OMe modified nucleotide
gcggucauugucacuggucuu

SEQ ID NO: 3 - DNA, 4001 bp, Homo sapiens
tccctctcccaccaggggtatacatcctcagagctgacccacgacctagctttctggtgtgactcggggtgggggctcccactggtcacctggtgacccccatcgcagtgagttccgccccaaggggaagcccagcctatagcaggctggggtggggtgtgtgcggagggaggtgggagaggcgtggaactagagaccctccaccttcatgtagaactaggggaacaaccttaggttccaagccccaagtccctatgtttccacccctttctaaggacaggcgtggaggagcggctggggctggcgggcttgtcgggatctcagctccctgagccctcctcctgccacgggcctgctcccctccttctctcatgggggtctgctgtagcctcgggaaggaggcaggaaacctccaaataaaatgacaaggcacgatttgctccccctactcagtaggcattggagcggtgagtttgcatttccaaggcacaaggttatcctaaatactagagttgccgggctcccagctcagccccaagaattctcccctcctcgcagggagaagccaccgcctggccccctcatcttagacgcaccaagtccggcgcagaggaagggaggggacacgcggagcaggccaggctttcaggaggcaccggaatctcctagtcctggctcgcacggctcgggcaagcctcgagatccggcgaccccaaaccactccctgggtccccgccggaggctggcccagggcggtcccacagccgcgcgcctcacgcgcagttgcccatggccttgaccaaggagctctctggcagctggcggaagatgccccgcagcgtgtccagttcgcggctcagctgttccacccgcttgcgcaggcggtcattgtcactggtcagctccagcaccttctgctgcgtctccacgttgcgctgcttggccttgtcgcggctcttgcgcaccgcgatgttgttgcgctcgcgccgcacccggtactcgttgctgttcttgtccaccgacttcttggccttgcccgcgccgctgccgccactcgcgcggaggtcggggtgcgcggcgcccagccccttgagcgcgctgccagggcccggcaggccggcggcaccgagcgcgggcgcggggtgcgggctgggcacgggcgtgggcggcggcgtggggtgaccgggctgcaggtgcatggtggtctggccgcagtgcgcgatctggaactgcaggtgcggggcggccaggtgcgcgggcggcgggtgcgggtgcgggtgcgagggcggcggcggcggcggcggctggtaagggaagaggccggccagcgccagctgcttggcttcatcctcctcgcggggctcctgcttgatcaccagcggccgcagcgccggcgccccgacgcgctcgtacaggggctccagcctgccgtccaggtagccggcggccgcgcagccgtagccgggcgggggcccgtgcgctcccccgggcatgacggcgccgccggggcccgcgggcgcgcccgggtagtcaaagtcgccgccgccgccgccgcccgtggggcccacggccgccttggccttctcctgctgccggctgtgctggaacaggtcggccaggaactcgtcgttgaaggcggccgggtcgatgtaggcgctgatgtcgatggacgtctcgtgctcgcagatgccgcccagcggctccggggcggcaggtggggcgggaggctgcgcggggcccgcgccccggggaaagccgaaggcggcgctgctgggcgcgtgcggggggctctgcaggtggctgctcatcgggggccgcggctccgcctcgtagaagtcggccgactccatgggggagttagagttctcccggcatggcgagcctcggcggcctccagcctgcgcggggcgtcgccgccgcccacccggagaccctgctcgcccgcgcccgcgcacctccgggtcgcgaatggcccggcccgcgccggcccagcttttatacccggcaggccgcgtcgccccctagagtccgaggcggcctctgtccccgggctgcggcggcgcggcgcctgctgggtcctagcgcgcggccggcatggggcggcgaaccagcgcggcacagcgccgcgctccccaggcaggccgcggcgcaacgcccaccgcctccagcgcgcccagcagagccgcggcgctcgctccaagctccgcccccggcccggccgtcgcccccgcgcccacgtggtcggtagcgggggccccctcctcctgcctgccctaggcgcccgtatccagccacggccgggagcccaggagtatcccgaggctgcacggggtaggggtggggggcggagggcgagtcttggtcttgagctgctggggcgcggattctctttcaaagccagaaccaggcctgtcccggacccgcgtcccggggaggctgcagcgcagagcagcggggctggggccggtggggggccgtttgggacgcgcggagaggtcctgagcgcggtggctctgcgtctcctagctctgatctccaggctacccctgtgattccgcgcagaggtacctctcggaggacgccggggtcccatgggcggcgccgcgcagggcgctaggaccccgcggggagcggaggcggcctcggcccgggagcctggaggacctggccggtcgatccgcccgggctggaaaactttctttataattacttctccaggtcggagcgcgcggcttgctaggcgcgcggggccggcgctgttacccggcgtggagtcgccgattttttttcctgcgggaccgcggggccccccagactagcggagctggacgccggggcgagcacggggaggggcgcaccgagggaggagacaaacttaactctggggccgggattccgaggcgggggccgcagccctcgaggcccgaagccaccgcttcctcccccgcctccccattcaggtgggcgccaacggcgggagcgagggtgtccaggccgccgggctgccaggtccgagcacgcacagggagaactctgcccagtggttcgccgggcgctgtagtccccgggatcctagggaccgaggcggccaggccctggggccccttgagtgcggcagctaatgctctcaccgcggcgggggaaggagcttgccaccgagacccccagccacgtgcgtccctcgcattctttaccggggccggggtggcggctacggaccgtcagctgggcccagatggagtcttgggagccctcaagtgtctcctgtccttgcccgcgccgcccctcgccactggcgctgaggcctgacgccgcctgcgtcccggctagaggcgcgcttgcctacaggtgagggaagacccccttcaccgacagtggccttaggcctggcaaggcgccacgacccgcccaggagccccggagggggcacagctaaaaacaccgctggagagccccgagcttccacgacgatcgcagtaaagaagcagtttcatctgggcaacgcacactgcgctttaatcaagttcctattcaacatagtcccagtgattaatagcccaactgcttcgttttcggtccagagctcataaacaagatatttttagcttgacgcttttggacgggagggagtaaaaaccagatacgttaaataaatatcccgatgtgagccggagagctgcttgctgagccaaatgcaggacccattcatatagcattcacctgtggagggagacctggacggaaatcaaaaagcaccaagagcgatttgcgtttttttctgcggtgctaaaactaatggcttttcctacctaggaacaaagaaacgccactgtacatgcacggttcccggcctgtggagttgtgggaggaaggcgatgtctggccttttttgcacagctgctgttgcctgcccagagatcgggaactctgccccgtaggactggaagaaacctcagtaatgggaataagactttgtccaatagggggctgatgaatgtgtg